SEQUENCE LISTING (23 sequences)

SEQ ID NO: 1 - protein, 6 aa, Homo sapiens
VQIVYK

SEQ ID NO: 2 - protein, 6 aa, Homo sapiens
VQIINK

SEQ ID NO: 3 - protein, 6 aa, synthetic construct
  SITE 1: note = D-Valine
VQIINK

SEQ ID NO: 4 - protein, 6 aa, synthetic construct
  SITE 3: note = D-Isoleucine
VQIINK

SEQ ID NO: 5 - protein, 6 aa, synthetic construct
  SITE 4: note = D-Isoleucine
VQIINK

SEQ ID NO: 6 - protein, 6 aa, synthetic construct
  SITE 1: note = D-Valine
  SITE 3: note = D-Isoleucine
VQIINK

SEQ ID NO: 7 - protein, 6 aa, synthetic construct
  SITE 1: note = D-Valine
VQIVYK

SEQ ID NO: 8 - protein, 6 aa, synthetic construct
  SITE 3: note = D-Isoleucine
VQIVYK

SEQ ID NO: 9 - protein, 6 aa, synthetic construct
  SITE 4: note = D-Valine
VQIVYK

SEQ ID NO: 10 - protein, 6 aa, synthetic construct
  SITE 5: note = D-Tyrosine
VQIVYK

SEQ ID NO: 11 - protein, 6 aa, synthetic construct
  SITE 6: note = D-Lysine
VQIVYK

SEQ ID NO: 12 - protein, 6 aa, synthetic construct
  SITE 1: note = D-Valine
  SITE 3: note = D-Isoleucine
VQIVYK

SEQ ID NO: 13 - protein, 6 aa, synthetic construct
  SITE 1: note = D-Valine
  SITE 5: note = D-Tyrosine
VQIVYK

SEQ ID NO: 14 - protein, 6 aa, synthetic construct
  SITE 3: note = D-Isoleucine
  SITE 5: note = D-Tyrosine
VQIVYK

SEQ ID NO: 15 - protein, 6 aa, synthetic construct
  SITE 4: note = D-Valine
  SITE 6: note = D-Lysine
VQIVYK

SEQ ID NO: 16 - protein, 6 aa, synthetic construct
  SITE 1: note = D-Valine
  SITE 3: note = D-Isoleucine
  SITE 5: note = D-Tyrosine
VQIVYK

SEQ ID NO: 17 - protein, 17 aa, synthetic construct
  SITE 8: note = D-Alanine
  SITE 11: note = D-Alanine
THKLTFRASHAVQIVYK

SEQ ID NO: 18 - protein, 17 aa, synthetic construct
  SITE 8: note = D-Alanine
  SITE 11: note = D-Alanine
THKLTFRASHAVQIINK

SEQ ID NO: 19 - protein, 6 aa, synthetic construct
THKLTF

SEQ ID NO: 20 - protein, 6 aa, synthetic construct
QVEVKS

SEQ ID NO: 21 - protein, 6 aa, synthetic construct
LDLSNV

SEQ ID NO: 22 - protein, 5 aa, synthetic construct
  SITE 2: note = D-Alanine
  SITE 5: note = D-Alanine
RASHA

SEQ ID NO: 23 - protein, 6 aa, synthetic construct
VPIVYK

(19) United States
(12) Patent Application Publication
(10) Pub. No.: US 2023/0399361 A1
(43) Pub. Date: Dec. 14, 2023

(54) MACROCYCLIC PEPTIDES

(71) Applicant: UNIVERSITY OF NOTRE DAME DU LAC, South Bend, IN (US)

(72) Inventors: Juan DEL VALLE, South Bend, IN (US); Benjamin Howard RAJEWSKI, South Bend, IN (US); Kamlesh MAKWANA, South Bend, IN (US)

(21) Appl. No.: 18/334,732

(22) Filed: Jun. 14, 2023

Related U.S. Application Data
(60) Provisional application No. 63/366,334, filed on Jun. 14, 2022.

(51) Int. Cl. C07K 7/54; A61K 38/17; A61K 38/08

(52) U.S. Cl. CPC C07K 7/54; A61K 38/1716; A61K 38/08

(57) ABSTRACT

Here, we describe a minimalist approach to mimic the aggregation-prone modules within tau. We carried out a backbone residue scan and showed that amide N-amination completely abolishes the tendency of these peptides to self-aggregate, rendering them soluble mimics of ordered β-strands from the tau R2 and R3 domains. Several N-amino peptides (NAPs) inhibit tau fibril formation in vitro. We further demonstrate that NAPs 12 and 13 are effective at blocking the cellular seeding of endogenous tau by interacting with monomeric or fibrillar forms of extracellular tau. Peptidomimetic 12 is serum stable, non-toxic to neuronal cells, and selectivity inhibits the fibrilization of tau over Aβ₄₂. Structural analysis of our lead NAPs shows considerable conformational constraint imposed by the N-amino groups. The described backbone N-amination approach provides a rational basis for the mimicry of other aggregation-prone peptides that drive pathogenic protein assembly.

### RELATED APPLICATIONS

This application claims priority under 35 U.S.C. § 119(e) to U.S. Provisional Patent Application No. 63/366,334, filed Jun. 14, 2022, which is incorporated herein by reference.

### GOVERNMENT SUPPORT

This invention was made with government support under Grant R01 AG074570 awarded by the National Institutes of Health. The government has certain rights in the invention.

### REFERENCE TO SEQUENCE LISTING

The present application includes a Sequence Listing in electronic format as an xml file titled “501.083US1_SL” which was created on Jun. 13, 2023 and has a size of 30,633 bytes. The contents of xml file 501.083US1_SL are incorporated herein by reference.

### BACKGROUND OF THE INVENTION

The higher-order assembly of proteins rich in β structure is correlated with poor prognosis in several neurodegenerative diseases. Intracellular accumulation of the tau protein into neurofibrillary tangles (NFTs) is linked to cognitive dysfunction in over 20 disorders collectively termed “tauopathies”. The normal function of tau is to stabilize microtubules (MTs), the support structures in axons. Pathogenic misfolding and aggregation can be caused by mutations in the MAPT gene that encodes for tau or by aberrant post-translational modifications. While toxicity has been associated with various forms of aggregated tau, current data support oligomeric species as a primary driver of neuronal death. It is now accepted that tau pathology becomes self-perpetuating, with the capacity to spread from neuron to neuron and cause normal tau to become misfolded. Controlling the processes that govern tau fibrilization and cellular propagation is critical for understanding the progression of tauopathies.

Tau is an intrinsically disordered protein harboring up to four MT-binding repeat domains (R1-R4) in the C-terminal half Like many amyloidogenic proteins, tau fibrilization involves conformational reorganization into β-rich folds, followed by supramolecular assembly into layered parallel β-sheets. This assembly is driven by favorable H-bonding and hydrophobic interactions between well-defined aggregation-prone hexapeptide motifs in the R2 (₂₇₅VQIINK₂₈₀; PHF6* (SEQ ID NO: 2)) and R3 (₃₀₆VQIVYK₃₁₁; PHF6 (SEQ ID NO: 1)) domains, which are also essential for MT binding. Short peptide models have long been used to study the structure and function of tau aggregates in vitro. However, direct inhibitors of tau fibrilization are largely limited to dyes and other redox-active aromatic compounds. Conformationally rigid and proteolytically stable peptidomimetics may hold particular promise as ligands of tau and other amyloidogenic proteins that are difficult to target in a sequence-specific manner. The aggregation-prone R2/R3 segments have more recently been used in the structure-based design of modified peptides that inhibit the aggregation of a PHF6 hexapeptide or truncated forms of recombinant tau. One group recently described a series of peptides capable of blocking the aggregation of full-length tau, as well as seeding in cells (Nat. Chem. 2018, 10, 170).

Despite examples of peptidomimetic disruptors of β-sheet-mediated protein interactions, strategies to translate conformationally extended peptide leads into inhibitors remain limited. This is due in part to the flexibility and proteolytic instability of short-peptide sequences, coupled with the large surface areas and diverse interaction modes associated with β-sheets. The propensity for conformationally extended peptides to aggregate via exposed H-bonding edges presents another significant challenge in the design of soluble β-strand mimics. Presentation of a β-strand epitope for protein recognition typically relies on the templating effect of an auxiliary β-strand (as in linear and macrocyclic β-hairpins), intra-strand conformational restriction through covalent tethering, or backbone amide N-alkylation to preclude strand self-aggregation. While backbone amide substitution allows for preservation of side chain functionality, N-methylation (or incorporation of Pro) can promote main chain torsions incompatible with β-sheet mimicry. So-called “β-breaker” peptidomimetics featuring amide substitutions have previously been developed to target β-amyloid assembly; however, there are currently no such examples capable of blocking the fibrilization or propagation of full-length tau. Despite numerous studies employing PHF6 and other truncated forms of tau to screen for new ligands, these model systems lack the residues required to adopt disease-associated folds and are incapable of seeding tau aggregation in cells.

Effective treatments of tauopathies are lacking. Accordingly, there is a need for biological agents and treatments to study and treat cognitive disorders.

### SUMMARY

We recently described an approach to β-strand stabilization based on peptide backbone N-amination (Angew. Chem., Int. Ed. 2017, 56, 2083). The conformational and non-aggregating characteristics of N-amino peptides (NAPs) are consistent across distinct models of β-sheet folding and are attributed to cooperative non-covalent interactions involving the Nα-NH₂ substituent. These qualities distinguish NAPs from N-alkylated peptides that do not readily adopt or stabilize β-strand conformations. Here, we describe the design and synthesis of NAPs that block tau fibrilization and seeding. Our N-amination strategy enables the use of a tau filament structure to guide the design of its own peptidomimetic ligands. Using biophysical and cellular propagation assays, we demonstrate the utility of a minimalist β-strand mimicry approach to target β-rich amyloids.

Accordingly, this disclosure provides an N-amino peptide comprising a paired helical filament hexapeptide (PHF6) wherein one or more amino acid moieties of the PHF6 have an amide nitrogen atom along the hexapeptide backbone that is N-aminated, wherein the N-aminated amide nitrogen atom is hydrazide moiety NNH₂.

This disclosure also provides a macrocyclic peptide comprising:
the N-amino peptide described above;
a second hexapeptide configured for forming a cross-beta structure;
a beta-arc configured for an antiparallel beta-hairpin turn wherein the beta-arc is covalently linked at one end to the PHF6 and the beta-arc is covalently linked at another end to the second hexapeptide; and
a di-cysteine linker (DCL) wherein the di-cysteine linker is covalently bonded to the sulfur atoms of two cysteine moieties, wherein one cysteine moiety is conjugated via a glycine moiety to the PHF6 and the second cysteine moiety is conjugated via another glycine moiety to the second hexapeptide to complete the macrocycle of the macrocyclic peptide.

Additionally, this disclosure provides a method for inhibiting tau fibrilization comprising contacting an effective amount of an N-amino peptide described above, and tau proteins comprising pathogenic tau fibrils, wherein the N-amino peptide blocks cellular transmission of pathogenic tau fibrils to the tau proteins and inhibits tau fibrilization of the tau proteins caused by the pathogenic tau fibrils.

The invention provides novel peptides of Formula I and Formula II, intermediates for the synthesis of peptides of Formula I and Formula II, as well as methods of preparing peptides of Formula I and II. The invention also provides peptides of Formula I and II that are useful as intermediates for the synthesis of other useful peptides. The invention provides for the use of peptides of Formula I and Formula II for the manufacture of medicaments useful for the treatment of cognitive disorders in a mammal, such as a human.

The invention provides for the use of the compositions described herein for use in medical therapy. The medical therapy can be treating a cognitive disorders, for example, Alzheimer's disease. The invention also provides for the use of a composition as described herein for the manufacture of a medicament to treat a disease in a mammal, for example, Alzheimer's disease in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Inhibition of tau_{P301L} aggregation and monomeric nature of inhibitors examined using ThT fluorescence: (A) Incubation of 20 μM inhibitors with 10 μM heparin-treated tau_{P301L} significantly reduced ThT fluorescence. (B) N-Amino substitution completely abolished the aggregation propensity of AcPHF6 and AcPHF6*, as determined by negligible ThT fluorescence after 48 h.

FIG. 2. Fibril morphology under a transmission electron microscope. Left to right: Incubation of 10 μM tau_{P301L} resulted in large, mature, and filamentous fibrils only in the presence of heparin. Addition of 20 μM NAP inhibitors 4, 12, and 13 afforded non-fibrillary amorphous aggregates similar to control. Scale bar represents a distance of 500 nm in the tau+hep panel and 2 μm in the other panels.

FIG. 3. Inhibition of tau aggregation and seeding. Soluble monomeric tau_{P301L} (0.19 μM) in the presence of heparin was co-incubated with 0.009 μM or 1.9 μM inhibitors for 4 days and then added to HEK293 cells stably expressing tau-RD (P301L/V337M)-YFP. Fibrillar tau_{P301L} can induce the aggregation of endogenous tau in cells seen as focal puncta with high fluorescence. Shown here are representative images of cells, at 20× magnification under FITC channel. Treatment with 1.9 μM of either 12 or 13 was sufficient to prevent seeding; however, 4 was found to be ineffective. Scale bar represents 200 μm. Bar graphs show the number of intracellular fluorescent puncta relative to control infection wells lacking the inhibitor.

FIG. 4. Concentrations of NAP inhibitors required to block seeding by pre-formed 0.19 μM tau_{P301L} fibrils in HEK293 cells expressing tau-RD (P301L/V337M)-YFP. Mature fibrils were treated with various concentrations of NAPs for 36 h, added to cells, and incubated for an additional 48 h. Punctate fluorescence was quantified to derive % tau infection relative to fibrils untreated with NAPs.

FIG. 5. Serum stability and cytotoxicity of NAP inhibitors: (A) NAPs 12 and 13 remained >80% intact after 24 h of incubation in 25% human serum, whereas the control peptide (H-LITRLENT-NH₂) was rapidly degraded, as measured by HPLC. (B) NAPs 12 and 13 did not exhibit any appreciable toxicity toward human neuro-blastoma (SH-SY5Y) cells at low (10 μM) or high (50 μM) concentrations after 48 h, as determined by MTT assay.

FIG. 6. ThT fluorescence assay with Aβ₄₂ in the presence or absence of 12 showing no inhibitory effect on Aβ₄₂ aggregation.

FIG. 7. Tau_{P301L} (5 μM) aggregation in the presence of heparin (5 μM) and NAPs (10 μM) as monitored by ThT fluorescence.

FIG. 8. TEM images of heparin-induced tau_{P301L} (5 mM) in the presence of 10 mM mxyl-NAP1, mxyl-NAP2, and corresponding controls (scale bar=500 nm).

FIG. 9. Inhibition of tau_{P301L} (190 nM) seeding activity in HEK293 biosensor cells by 95 nM mxyl-NAP1, mxyl-NAP2, and corresponding control NAPs.

FIG. 10. Endogenous tau-RD(LM)-YFP aggregation seeded by 190 nM tau_{P301L} in the presence or absence of 95 nM mxyl-NAPs. Representative fluorescence microscopy images of HEK293 biosensor cells at 20× magnification under FITC channel (scale bars=200 nm).

FIG. 11. Dose-dependent anti-seeding activity of mxyl-NAP1 and mxyl-NAP2 against 190 nM tau_{P301L}.

FIG. 12. (A) Extraction and cell-based seeding assay with AD tau. Dose-dependent anti-seeding activity of (B) mxyl-NAP1 and (C) mxyl-NAP2 against patient-derived extracts of AD tau.

### DETAILED DESCRIPTION

The spread of neurofibrillary tangles composed of tau protein aggregates is a hallmark of Alzheimer's and related neurodegenerative diseases. Early oligomerization of tau involves conformational reorganization into parallel β-sheet structures and supramolecular assembly into toxic fibrils. Despite the need for selective inhibitors of tau propagation, β-rich protein assemblies are inherently difficult to target with small molecules.

Described herein is the design, synthesis, and biological evaluation of a novel class of β-strand mimics that block tau fibrilization and propagation. Since the physiological relevance of truncated tau fibrilization models has recently been called into question, the current study focused on targeting tau_{P301L}, which harbors cross-β residues that are important for adopting pathogenic folds. Using an amide-to-hydrazide replacement strategy, we carried out a positional scan of aggregation-prone peptide sequences derived from the R2 and R3 domains of tau. Several NAP analogues inhibited the fibrilization of recombinant full-length tau as well as its seeding capacity in an in-cell aggregation assay. Key features of the described NAP inhibitors include increased conformational rigidity, resistance toward self-aggregation, and remarkable stability toward serum proteases.

The cell-to-cell propagation of fibrillar tau has emerged as an important target for structure-based ligand design. The majority of small-molecule anti-tau approaches have relied on modulating disease-associated post-translational modifications, targeting intracellular tau to prevent aggregation, or disaggregating mature fibrils. Ligands such as 12 can block the seeding capacity of extracellular tau fibrils, thus enabling studies on the role that propagation plays in the progression of tauopathies. Importantly, it is estimated that over 99% of endogenous tau is bound to microtubules leaving only a small fraction as free tau within neurons. Extracellular seed-competent tau is estimated to exist in only picomolar or low nanomolar concentrations in the human brain, even in a pathological state. Ligands that require stoichiometric quantities to block tau fibril transmission therefore represent viable chemical probes of propagation in cells and in vivo.

Seminal work from the laboratories of Doig (J. Biol. Chem. 2000, 275, 25109) and Meredith (Biochemistry 2001, 40, 8237) demonstrated the utility of peptide backbone N-methylation for the development of inhibitors of Aβ aggregation. Derived from aggregation-prone β-strand sequences, these and related β-breaker peptidomimetics feature backbone substitutions designed to interrupt canonical β-sheet H-bonding patterns on one edge. Although N-methylated PHF6 analogues with anti-aggregation activity against tau are not known, Nowick and co-workers (J. Am. Chem. Soc. 2011, 133, 3144) reported β-hairpin macrocycles that bind to AcPHF6 via a recognition strand while inhibiting higher-order assembly. More recently, Segal and co-workers (Chem. Eur. J. 2017, 23, 9618) developed a proline-containing analogue of PHF6 (Ac-VPIVYK-NH₂ (SEQ ID NO: 23)) that inhibits the aggregation of AcPFH6 in vitro. Structure-based side chain mutational strategies and phage display selection have been employed to identify larger peptide-based inhibitors of tau with cellular and in vivo efficacy. To our knowledge, NAP-based tau ligands such as 12 and 13 represent the first PHF6 peptidomimetics capable of blocking the fibrilization and cellular propagation of full-length tau.

In using the sequence of tau to guide the design of its own inhibitors, our work also sets the stage for the development of ligands that are selective for other pathogenic amyloids. Our most effective inhibitor of tau fibrilization and seeding showed no effect on the in vitro aggregation of Aβ₄₂. Discrimination between structurally related β-rich assemblies is potentially enabled by NAPs, which exhibit a full complement of side chains in a minimalist single-strand design. Given that disease-associated conformational strains of tau are known to propagate in vivo with high fidelity, we expect that an NAP-based strategy may be used to target unique structural motifs within such polymorphs.

Additional information and data supporting the invention can be found in the following publication by the inventors: ACS Chem. Neurosci. 2021, 12, 20, 3928-3938 and its Supporting Information, which are incorporated herein by reference in its entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modern Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom or two different carbon atoms of a carbon chain. Similarly, alkenylene and alkynylene are respectively an alkene and an alkyne having two free valences at two different carbon atoms, or an alkenylene can have the two free valences on the same carbon.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3- to 10-membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents R^{X} where R^{X} is at the ortho-, meta-, or para-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents of the indicated groups can be those recited in a specific list of substituents described herein, or as one of skill in the art would recognize, can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents of indicated groups can be bonded to a substituted carbon atom include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as O, it is bonded to the atom it is substituting by a double bond; for example, a carbon atom substituted with O forms a carbonyl group, C═O.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof, such as racemic mixtures, which form part of the present invention. Many organic compounds exist in optically active forms, i.e., they have the ability to rotate the plane of plane-polarized light. In describing an optically active compound, the prefixes D and L, or R and S. are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate (defined below), which may occur where there has been no stereoselection or stereospecificity in a chemical reaction or process.

The term “IC₅₀” is generally defined as the concentration required to inhibit a specific biological or biochemical function by half, or to kill 50% of the cells in a designated time period, typically 24 hours.

The term amino acids described herein are among the 20 naturally occurring amino acids that have the L-configuration unless indicated otherwise. An amino acid that has the D-configuration is denoted symbolically with a superscript capital “D” preceding its name or its standard abbreviation. For example, the D-configuration of alanine with be abbreviated as ^{D}alanine ^{D}Ala, or ^{D}A; and it may also be indicated as D-alanine or a lower case single letter abbreviation, e.g., “a” for D-alanine.

### Embodiments of the Technology

1. An N-amino peptide comprising a paired helical filament hexapeptide (PHF6) wherein one or more amino acid moieties of the PHF6 have an amide nitrogen atom along the hexapeptide backbone that is N-aminated, wherein the N-aminated amide nitrogen atom is hydrazide moiety NNH₂.
2. The N-amino peptide of embodiment 1 comprising Formula I:

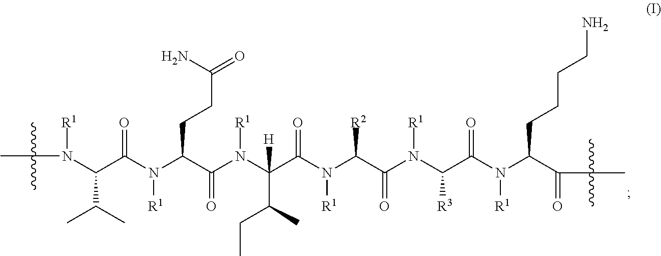

wherein
   each R¹ is independently H or NH₂ wherein at least one R¹ is NH₂;
   R² is isoleucine or valine; and
   R³ is asparagine or tyrosine.
3. The N-amino peptide of embodiment 1 or 2 wherein the PHF6 comprises -Val-Gln-Ile-Val-Tyr-Lys-(VQIVYK) (SEQ ID NO: 1) or -Val-Gln-Ile-Ile-Asn-Lys-(VQIINK) (SEQ ID NO: 2).
4. The N-amino peptide of any one of embodiments 1-3 wherein one, two, or three amino acid moieties of the PHF6 have an amide nitrogen atom along the hexapeptide backbone that is N-aminated, wherein the N-aminated amide nitrogen atom is the hydrazide moiety NNH₂.
5. The N-amino peptide of any one of embodiments 1-4 wherein the N-amino peptide has terminal ends that terminate with an amide moiety.
6. The N-amino peptide of embodiment 1 wherein the N-amino peptide is:

(1)
(SEQ ID NO: 3)
Ac-aVal-Gln-Ile-Ile-Asn-Lys-NH₂;
(2)
(SEQ ID NO: 4)
Ac-Val-Gln-aIle-Ile-Asn-Lys-NH₂;
(3)
(SEQ ID NO: 5)
Ac-Val-Gln-Ile-aIle-Asn-Lys-NH₂;
(4)
(SEQ ID NO: 6)
Ac-a Val-Gln-aIle-Ile-Asn-Lys-NH₂;
(5)
(SEQ ID NO: 7)
Ac-aVal-Gln-Ile-Val-Tyr-Lys-NH₂;
(6)
(SEQ ID NO: 8)
Ac-Val-Gln-aIle-Val-Tyr-Lys-NH₂;
(7)
(SEQ ID NO: 9)
Ac-Val-Gln-Ile-aVal-Tyr-Lys-NH₂;
(8)
(SEQ ID NO: 10)
Ac-Val-Gln-Ile-Val-aTyr-Lys-NH₂;
(9)
(SEQ ID NO: 11)
Ac-Val-Gln-Ile-Val-Tyr-aLys-NH₂;
(10)
(SEQ ID NO: 12)
Ac-aVal-Gln-aIle-Val-Tyr-Lys-NH₂;
(11)
(SEQ ID NO: 13)
Ac-aVal-Gln-Ile-Val-aTyr-Lys-NH₂;
(12)
(SEQ ID NO: 14)
Ac-Val-Gln-aIle-Val-aTyr-Lys-NH₂;
(13)
(SEQ ID NO: 15)
Ac-Val-Gln-Ile-aVal-Tyr-aLys-NH₂;
or
(14)
(SEQ ID NO: 16)
Ac-aVal-Gln-aIle-Val-aTyr-Lys-NH₂;

wherein
   the prefix “a” (preceding an amino acid abbreviation) is an NH₂ moiety on an N-aminated amide nitrogen of the respective amino acid moiety of the N-amino peptide (1-14);
   Ac is the acetylated N-terminus of the N-amino peptide (1-14); and
      NH₂ is the amidated C-terminus of the N-amino peptide (1-14).

In some embodiments, the N-amino peptide is:

(12)
(SEQ ID NO: 14)
Ac-Val-Gln-aIle-Val-aTyr-Lys-NH₂;
or
(13)
(SEQ ID NO: 15)
Ac-Val-Gln-Ile-aVal-Tyr-aLys-NH₂.

7. The N-amino peptide of any one of embodiments 1-5 wherein the N-amino peptide comprises the peptide sequence:

(SEQ ID NO: 17)
-Tyr-His-Lys-Leu-Thr-Phe-Arg-^{D}Ala-Ser-His-^{D}Ala-
Val-Gln-Ile-Val-Tyr-Lys-(THKLTFR^{D}ASH^{D}AVQIVYK);
or
(SEQ ID NO: 18)
-Tyr-His-Lys-Leu-Thr-Phe-Arg-^{D}Ala-Ser-His-^{D}Ala-
Val-Gln-Ile-Ile-Asn-Lys-(THKLTFR^{D}ASH^{D}AVQIINK)

8. The N-amino peptide of any one of embodiments 1-5 or 7 wherein the N-terminus of the peptide sequence comprises the dipeptide -Cys-Gly-, and the C-terminus of the peptide sequence comprises the dipeptide -Gly-Cys-.
9. A macrocyclic peptide comprising:
   the N-amino peptide of embodiment 1;
   a second hexapeptide configured for forming a cross-beta structure;
   a beta-arc configured for an antiparallel beta-hairpin turn wherein the beta-arc is covalently linked at one end to the PHF6 and the beta-arc is covalently linked at another end to the second hexapeptide; and
   a di-cysteine linker (DCL) wherein the di-cysteine linker is covalently bonded to the sulfur atoms of two cysteine moieties, wherein one cysteine moiety is conjugated via a glycine moiety to the PHF6 and the second cysteine moiety is conjugated via another glycine moiety to the second hexapeptide to complete the macrocycle of the macrocyclic peptide.
10. The macrocyclic peptide of embodiment 9 wherein the second hexapeptide is:

(SEQ ID NO: 19)
-Thr-His-Lys-Leu-Thr-Phe-(THKLTF);
(SEQ ID NO: 20)
-Gln-Val-Glu-Val-Lys-Ser-(QVEVKS);
or
(SEQ ID NO: 21)
-Leu-Asp-Leu-Ser-Asn-Val-(LDLSNV).

11. The macrocyclic peptide of embodiments 9 or 10 wherein the beta-arc is the peptide moiety:

(SEQ ID NO: 22)
-Arg-^{D}Ala-Ser-His-^{D}Ala-(R^{D}ASH^{D}A).

12. The macrocyclic peptide of any one of embodiments 9-11 wherein the macrocyclic peptide has a C-terminus and an N-terminus, wherein both the C-terminus and the N-terminus terminate with an amide moiety.
13. The macrocyclic peptide of any one of embodiments 9-12 wherein the di-cysteine linker (DCL) is

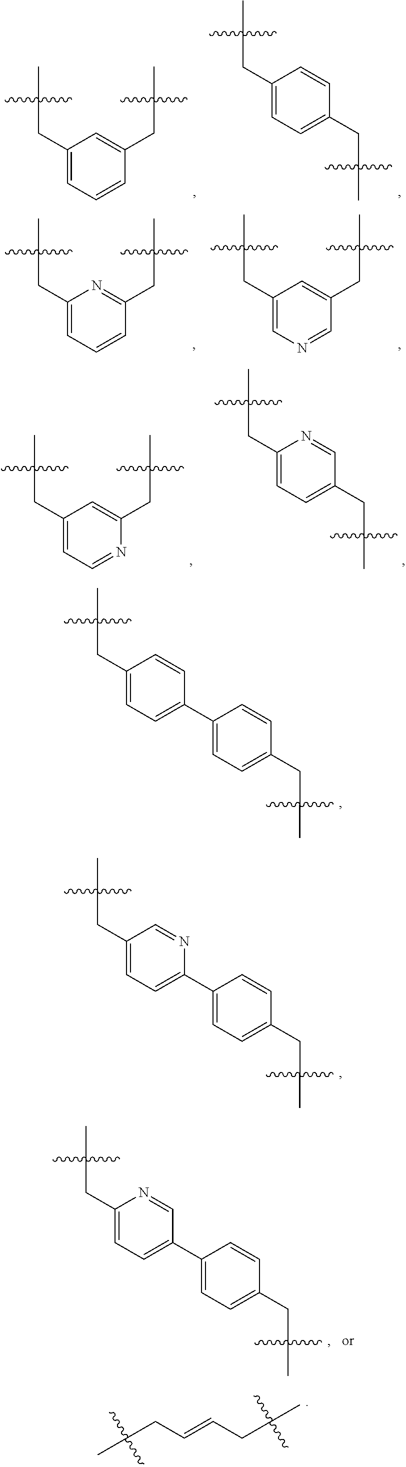

14. The macrocyclic peptide of any one of embodiments 9-13 represented by Formula II:

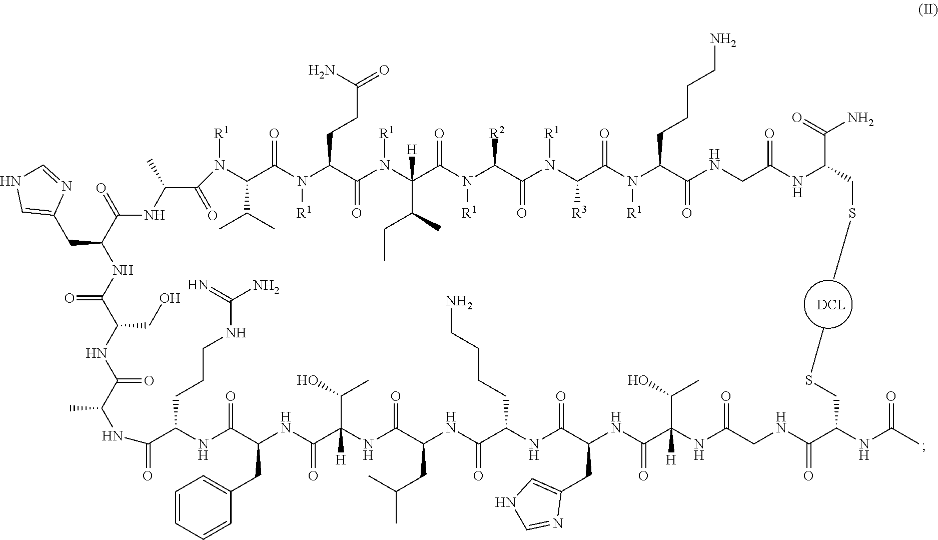

wherein
   each R¹ is independently H or NH₂ wherein at least one R¹ is NH₂;
   R² is isoleucine or valine;
   R³ is asparagine or tyrosine; and
   DCL is the di-cysteine linker.
15. The macrocyclic peptide of any one of embodiments 9-14 wherein DCL is

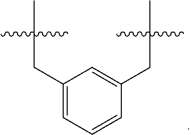 [SMILES: *Cc1cccc(C*)c1]

16. The macrocyclic peptide of embodiment 9 wherein the macrocyclic peptide is:

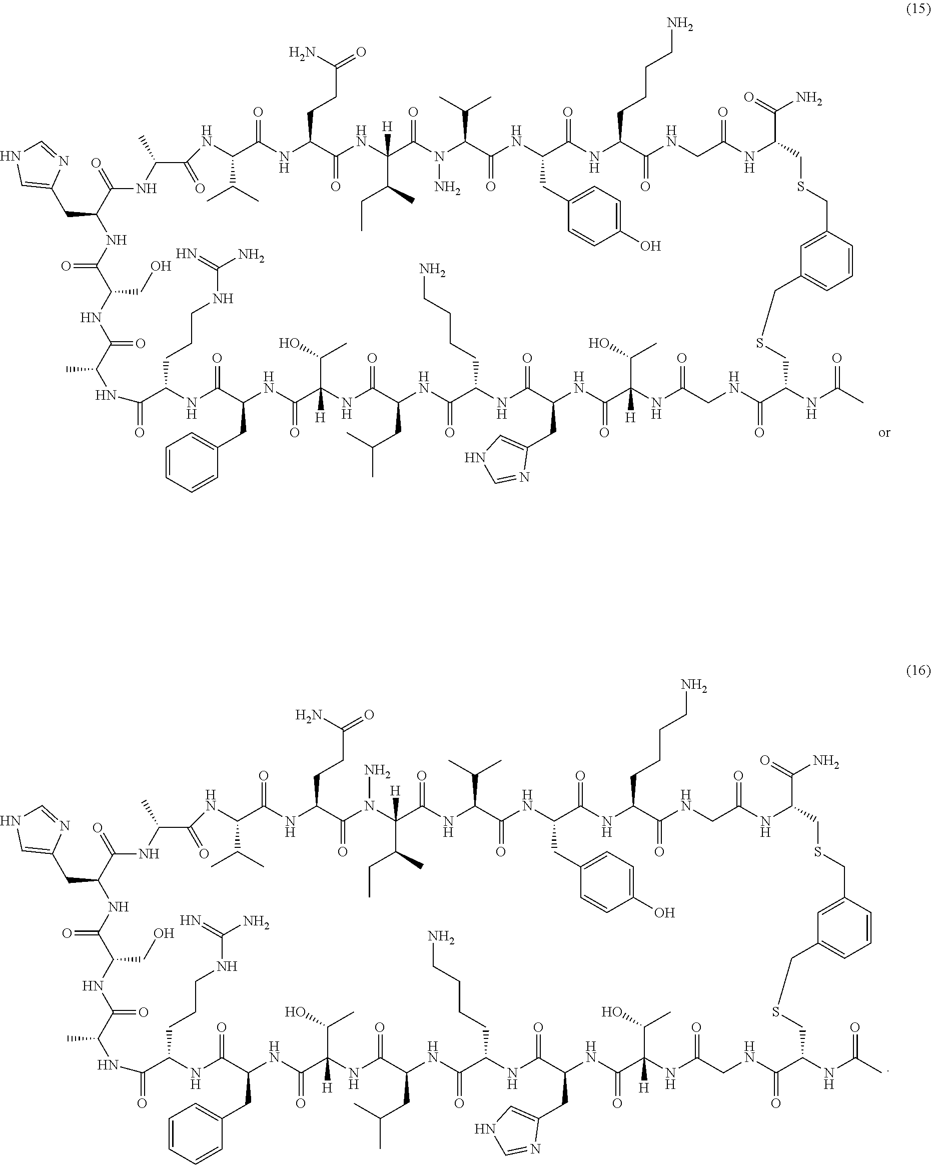

17. A method for inhibiting tau fibrilization comprising contacting an effective amount of an N-amino peptide of any one of embodiments 1-16 and tau proteins comprising pathogenic tau fibrils, wherein the N-amino peptide blocks cellular transmission of pathogenic tau fibrils to the tau proteins and inhibits tau fibrilization of the tau proteins caused by the pathogenic tau fibrils.
18. The method of embodiment 17 wherein an effective amount of the N-amino peptide is about 1 micromolar to about 100 micromolar. In some embodiments, the effective amount of the N-amino peptide is about 1 nanomolar to about 1000 micromolar. In some other embodiments, the effective amount of the N-amino peptide (in micromolar) is about 5, 10, 15, 20, 25, 30, 35, 40, 50, 75, 100, 150, 200, 300, 400, 500, 600, 700, 800, or about 1000 micromolar.
19. The method of embodiment 17 or 18 wherein the N-amino peptide is a macrocyclic peptide represented by Formula II:

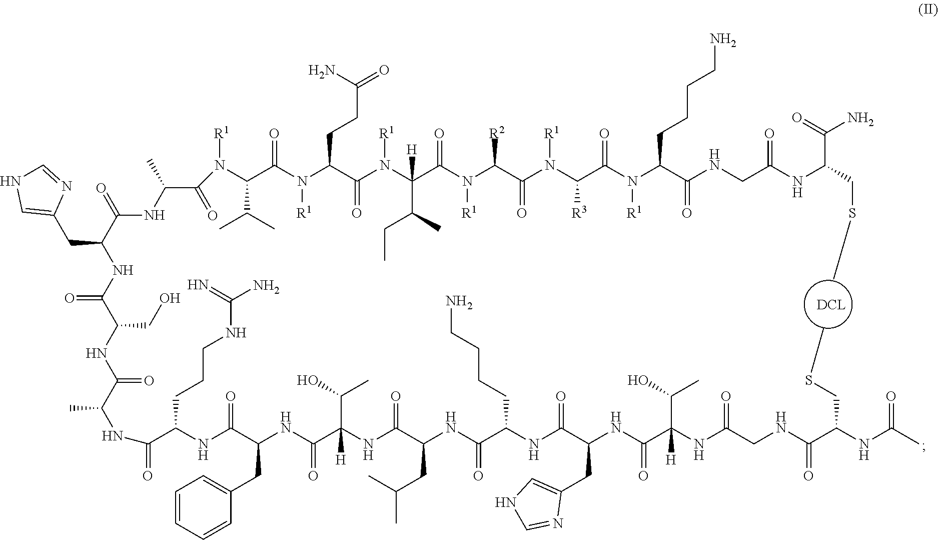

wherein
   each R¹ is independently H or NH₂ wherein at least one R¹ is NH₂;
   R² is isoleucine or valine;
   R³ is asparagine or tyrosine; and
   DCL is:

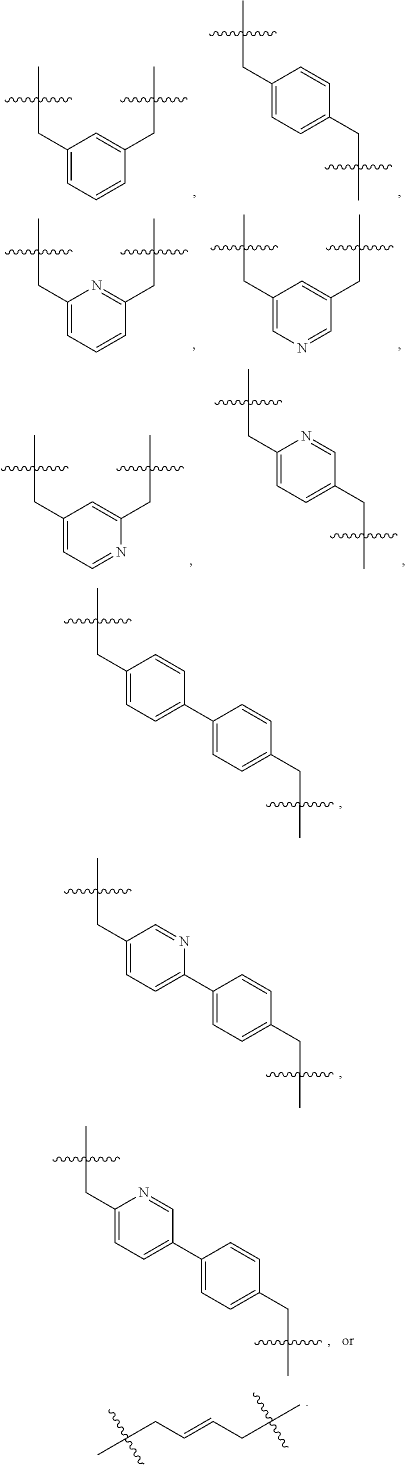

20. The method of any one of embodiments 17-19 wherein the macrocyclic peptide is:

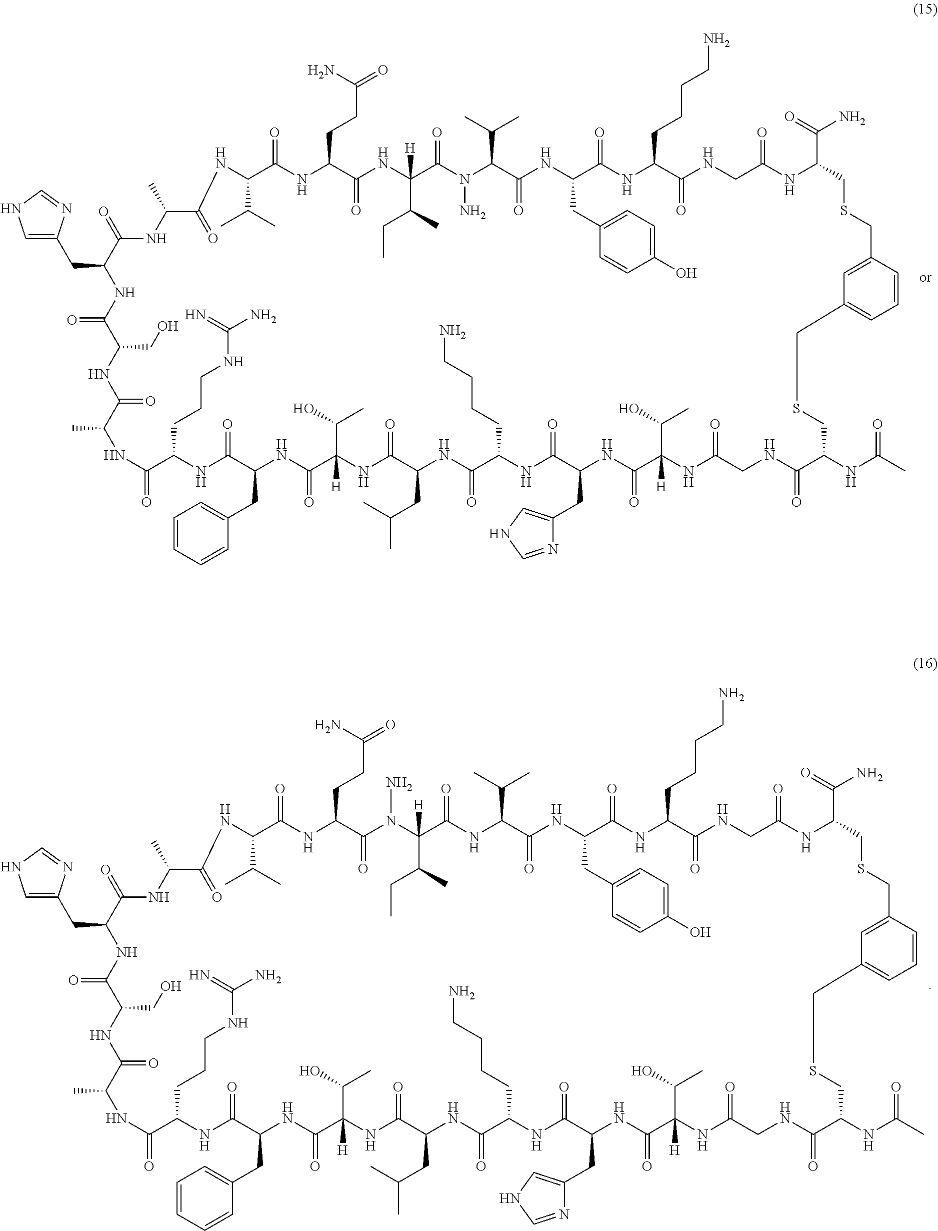

### Results and Discussion.

Design and Synthesis of NAP-Based Tau Ligands. The R3₃₀₆VQIVYK₃₁₁ (SEQ ID NO: 1) hexapeptide motif is widely accepted as the key amyloidogenic core of tau because filaments formed from this motif closely resemble those observed from Alzheimer's disease tau. However, recent crystal structures of the R2₂₇₅VQIINK₂₈₀ (SEQ ID NO: 2) motif show tighter side chain packing and strand interdigitation relative to the R3 hexapeptide, suggesting it to be a more powerful driver of tau aggregation. Since the specific contribution of individual residues in these sequences has not yet been studied, we chose to perform a complete backbone N-amino scan along the length of each hexapeptide. Our NAP-based library included mono-, di-, and tri-N-aminated analogues. We limited poly-N-amino peptides to those harboring amide substitutions on a single H-bonding edge, thus retaining a fully intact edge for interaction with tau. We synthesized 14 NAP β-strand mimics on a solid support, as described in Chart 1. We excluded analogues harboring N-amino glutamine (aGln) or N-amino asparagine (aAsn) residues since these undergo rapid intraresidue cyclization via the hydrazide during cleavage. Our strategy relied on the incorporation of orthogonally protected N-amino dipeptide building blocks that are available in three steps from the corresponding α-amino benzyl esters. Notably, this dipeptide fragment approach allows for Fmoc solid-phase peptide synthesis (SPPS) of NAPs using automated, microwave-assisted HCTU/NMM condensation protocols on a Rink amide MBHA resin. In contrast to canonical dipeptide (or larger) fragments, N-aminated building blocks are highly resistant to racemization during activation owing to the electron-withdrawing NHBoc substituent. Following elongation, NAPs were cleaved from the resin and purified by preparative RP-HPLC. All NAPs were characterized by ¹H NMR and HRMS. The parent unmodified hexapeptides AcPHF6 and AcPHF6* were also synthesized for comparison with backbone-aminated variants.

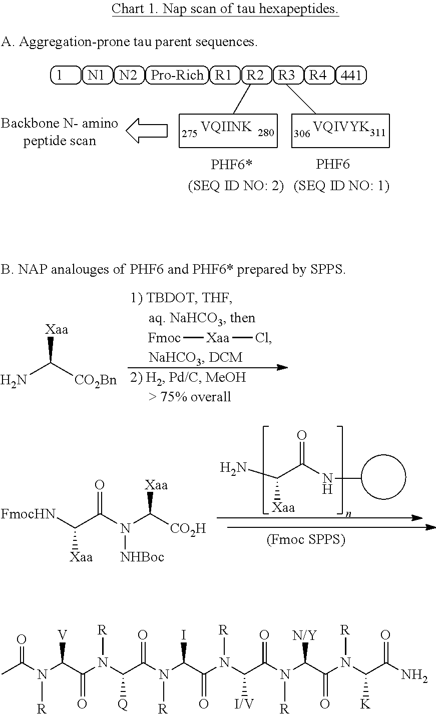

| Compound | Sequence | [M + H]⁺_{obs} | SEQ ID NO |
|---|---|---|---|
| 1 | Ac-aVal-Gln-Ile-Ile-Asn-Lys-NH₂ | 770.4877 | SEQ ID NO: 3 |
| 2 | Ac-Val-Gln-aIle-Ile-Asn-Lys-NH₂ | 770.4878 | SEQ ID NO: 4 |
| 3 | Ac-Val-Gln-Ile-aIle-Asn-Lys-NH₂ | 770.4879 | SEQ ID NO: 5 |
| 4 | Ac-aVal-Gln-aIle-Ile-Asn-Lys-NH₂ | 820.5045 | SEQ ID NO: 6 |
| 5 | Ac-aVal-Gln-Ile-Val-Tyr-Lys-NH₂ | 805.4930 | SEQ ID NO: 7 |
| 6 | Ac-Val-Gln-aIle-Val-Tyr-Lys-NH₂ | 80.4925 | SEQ ID NO: 8 |
| 7 | Ac-Val-Gln-Ile-aVal-Tyr-Lys-NH₂ | 805.4927 | SEQ ID NO: 9 |
| 8 | Ac-Val-Gln-Ile-Val-aTyr-Lys-NH₂ | 805.4927 | SEQ ID NO: 10 |
| 9 | Ac-Val-Gln-Ile-Val-Tyr-aLys-NH₂ | 805.4933 | SEQ ID NO: 11 |
| 10 | Ac-aVal-Gln-aIle-Val-Tyr-Lys-NH₂ | 820.5045 | SEQ ID NO: 12 |
| 11 | Ac-aVal-Gln-Ile-Val-aTyr-Lys-NH₂ | 820.5041 | SEQ ID NO: 13 |
| 12 | Ac-Val-Gln-aIle-Val-aTyr-Lys-NH₂ | 820.5048 | SEQ ID NO: 14 |
| 13 | Ac-Val-Gln-Ile-aVal-Tyr-aLys-NH₂ | 820.5039 | SEQ ID NO: 15 |
| 14 | Ac-aVal-Gln-aIle-Val-aTyr-Lys-NH₂ | 835.5115 | SEQ ID NO: 16 |
| AcPHF6* | Ac-Val-Gln-Ile-Ile-Asn-Lys-NH₂ | 755.4775 | SEQ ID NO: 2 |
| AcPHF6 | Ac-Val-Gln-Ile-Val-Tyr-Lys-NH₂ | 790.4833 | SEQ ID NO: 1 |

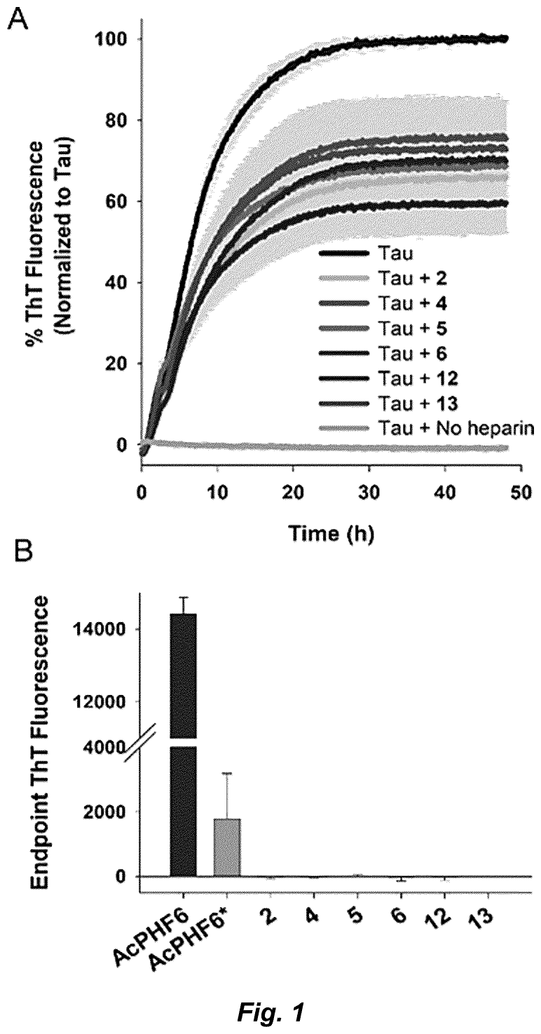

NAP Tau Mimics Inhibit Tau Fibrilization In Vitro. We chose thioflavin T (ThT), an amyloid specific fluorescent dye that binds to β-sheet assemblies, to first evaluate the effect of NAPs on recombinant tau aggregation. For these studies, we expressed and purified full-length tau featuring a P301L mutation frequently observed in patients with frontotemporal dementia and Parkinsonism linked to chromosome 17 (FTDP-17). This missense mutation leads to local structure destabilization around the amyloid-forming region resulting in faster aggregation. Recombinant tau_{P301L} aggregated in the presence of equimolar heparin sulfate (t_{1/2}=6.5±0.4 h) starting with a very short 0.5 h lag phase, followed by a 24 h exponential growth phase. Of the 14 NAPs tested, 6 were found to significantly reduce the endpoint ThT fluorescence of tau_{P301L} when incubated at 2-fold molar excess (FIG. 1A). Compounds 2 and 4 are mono- and diaminated hexapeptides derived from the R2 PHF6* aggregation-prone sequence, whereas compounds 5, 6, 12, and 13 are all derived from the R3 PHF6 sequence. Several other NAPs had no effect on endpoint ThT fluorescence or lacked consistent inhibition across repeated experiments. In agreement with previous reports, we observed significant aggregation of both the R2 and R3 parent peptides (AcPHF6* and AcPHF6) when incubated alone in aq. NaOAc buffer, as evidenced by intense ThT fluorescence after 48 h (FIG. 1B). In contrast, the six NAP inhibitors mentioned above (2, 4-6, 12, and 13) exhibited no such fluorescence, suggesting that even a single backbone N-amino group was sufficient to confer resistance to self-aggregation.

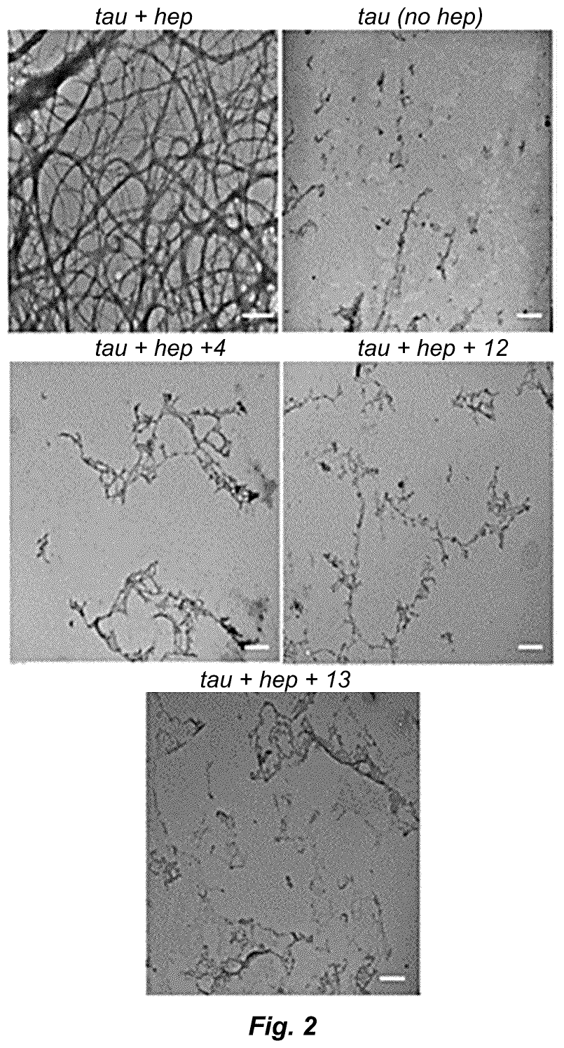

Although useful in identifying NAPs that have an effect on tau_{P301L} fibrilization, ThT fluorescence data suggested that the formation of tau oligomeric assemblies can still occur in the presence of NAPs. This is consistent with previous reports showing that the endpoint ThT fluorescence and fibrilization lag time for full-length tau are particularly resistant to perturbation by inhibitors. Several peptidomimetic and small-molecule tau aggregation inhibitors are thus highly effective in ThT assays using 3R or other truncated tau isoforms and less so in the presence of 4R tau. To confirm the effect of NAPs on the growth of mature tau fibrils, we used transmission electron microscopy (TEM) to visualize the morphology and maturity of fibrillar species. Heparin-induced tau_{P301L} fibrils were allowed to grow over 96 h in the presence or absence of inhibitors. Untreated tau_{P301L} afforded large, helical, and amyloid-like filamentous fibrils. In contrast, we did not observe mature fibril networks in the presence of a 2-fold molar excess of the six NAP inhibitors mentioned above. Di-N-aminated peptides 4, 12, and 13 were particularly effective at attenuating fibril growth, resulting in aggregates similar to control wells containing tau_{P301L} without heparin (FIG. 2). In the case of the mono-N-aminated peptides (2, 5, and 6), we observed short, immature rod-like fibrils, indicative of a more modest effect on tau assembly.

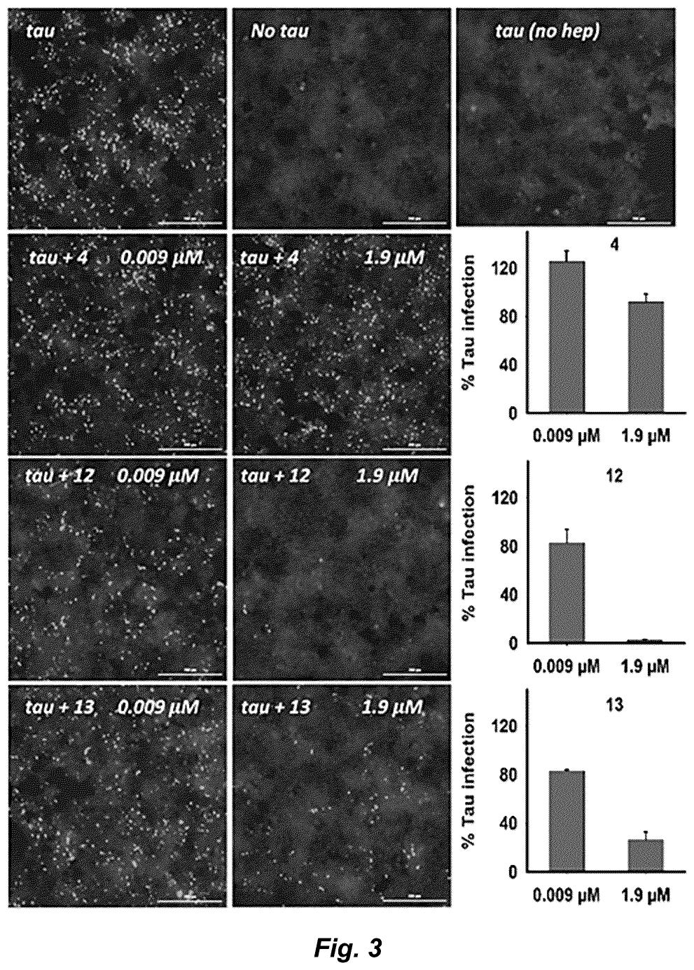

Di-N-aminated Hexapeptides Block the Cellular Transmission of Tau Fibrils. Recent studies show that extracellular tau fibrils spread in a prion-like fashion from one cell to the next. This mode of propagation is important for the spread of NFTs, neuropil threads, and plaque-associated neurites—all of which contribute to the progression of Alzheimer's disease. To test whether NAP inhibitors are able to block the seeding activity of recombinant tau_{P301L}, we employed HEK293 biosensor cells that stably express a tau-yellow fluorescent protein fusion [tau-RD(LM)-YFP]. When we treated these cells with preformed heparin-induced fibrils of tau_{P301L}, we observed a large number of intracellular tau aggregates, as indicated by punctate fluorescence after 48 h. These wells exhibited a mean of 38% aggregate-containing cells over three separate experiments, demonstrating the ability for fibrillar tau_{P301L} to enter cells and seed the aggregation of the endogenous tau-RD(LM)-YFP (FIG. 3). Given their superior anti-fibrillar activity by TEM and reduced peptide character, we elected to carry out cell-seeding experiments with di-N-aminated peptides 4, 12, and 13. Pre-treatment of monomeric tau_{P301L} with 1.9 μM di-NAPs 12 and 13 (derived from the R3 PHF6 motif) significantly reduced seeding capacity. This effect was less pronounced at 0.009 μM. Inhibitor 4, derived from the R2 domain PHF6* motif exhibited far weaker anti-seeding activity at both high and low concentrations (FIG. 3).

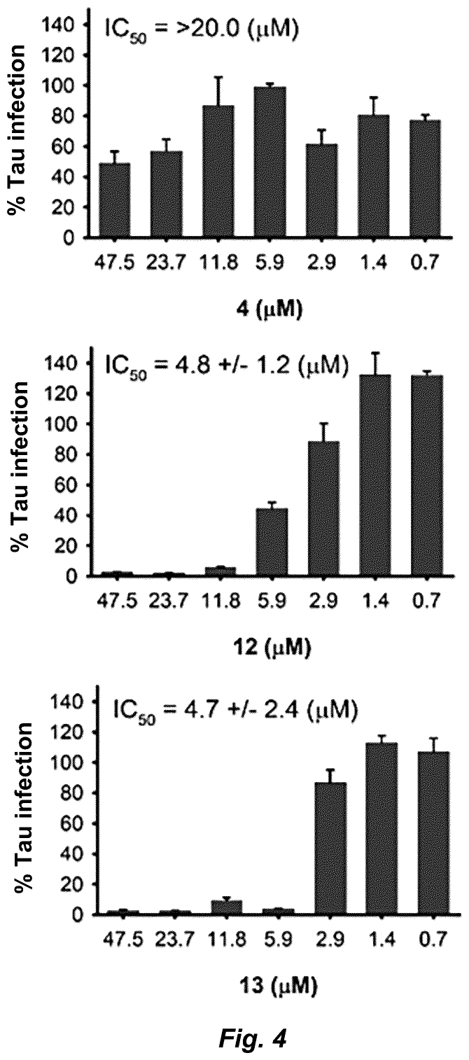

Given that pathogenic tau can be secreted from cells in various forms (as oligomers, aggregates, or mature fibrils), we tested whether NAPs could block the cellular transmission of pre-formed fibrils. In this experiment, NAPs were incubated with mature tau_{P301L} fibrils for 36 h prior to treatment of cells expressing tau-RD(LM)-YFP. Indeed, we found that com-pounds 12 and 13 were able to effectively inhibit propagation in a dose-dependent manner. We determined a fibril capping IC₅₀ in cells in the 5 μM range across three repeated experiments (FIG. 4). These results demonstrate that our structure-based NAP mimicry approach endows ligands with anti-seeding activity irrespective of the tau aggregation state.

Consistent with the seeding experiments performed above using monomeric tau_{P301L}, di-NAP 4 was generally ineffective at blocking the propagation of pre-formed fibrils (FIG. 4). We then considered the possibility of di-NAPs 12 and 13 entering cells and inhibiting seeding by interacting with endogenous tau-RD(LM)-YFP, rather than with extracellular pre-formed fibrils. We thus repeated the experiment without the 36 h inhibitor+mature fibril co-incubation period. Both di-NAPs 12 and 13 failed to inhibit endogenous tau aggregation in this experiment, suggesting that the compounds interact with extracellular tau_{P301L} to block cellular trans-mission.

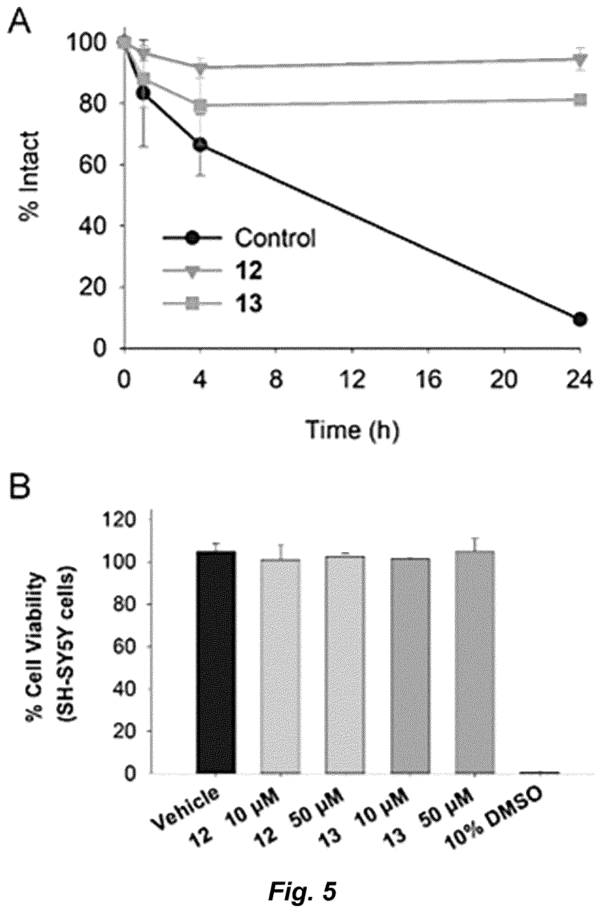

Di-NAPs Are Stable in Human Serum and Non-toxic to Neuronal Cells. Compounds 12 and 13 feature two hydrazide bonds within the peptidomimetic backbone. Their utility as tau ligands in cell-based experiments would benefit from resistance to proteolytic degradation. We carried out stability studies in human serum and monitored degradation by RP-HPLC (FIG. 5A). Both 12 and 13 were found to be remarkably stable in 25% human serum (>83% intact after 24 h). In contrast, an eight-residue control peptide was rapidly degraded over 24 h in the same assay. Although the parent AcPHF6 peptide could not be used as a control due to rapid self-aggregation, the stability of 12 and 13 demonstrates the ability of N-amination to protect against peptide backbone degradation.

Cellular seeding experiments with tau_{P301L} in the presence or absence of di-NAPs 12 and 13 did not result in detectable toxicity to HEK293 biosensor cells. We carried out MTT assays to ensure that inhibitors 12 and 13 are not toxic to human neural cells. As shown in FIG. 5B, 12 and 13 exhibited no appreciable toxicity toward SH-SY5Y (human neuroblastoma) cells up to 50 μM, or 10-fold their anti-seeding IC₅₀ values.

N-Amination Imparts Backbone Conformational Constraint in Solution. Di-NAPs that potentially cap mature tau fibrils would be expected to adopt parallel β-sheet-like conformations. X-ray crystallography of a model di-N-aminated tripeptide previously demonstrated its self-association as a dimeric species with extended backbone geometries. To gain an insight into the solution structure of our lead tau ligands, we carried out 2D-NMR spectroscopy, followed by simulated annealing. Although AcPHF6 was insoluble in water, we were able to obtain gradient correlation spectroscopy (gCOSY), total correlation spectroscopy (TOCSY), and rotating frame Overhauser enhancement spectroscopy (ROESY) NMR spectra in 9:1 H₂O/D₂O for 12 and 13. ¹H NMR spectra were remarkably well resolved and devoid of significant minor rotamers despite the presence of two N-substituted amide bonds. Moreover, inter-residue nuclear Overhauser enhancements (NOEs) were limited to correlations consistent with an extended solution conformation (CαHᵢ→NHᵢ₊₁). Though short linear peptides are expected to be highly flexible in solution, we observed no NOE correlations characteristic of turn conformations. Distance-restrained simulated annealing and clustering based on backbone dihedral angles afforded ensembles of the three most populated conformers of 12. These clusters revealed a high convergence of φ and ψ torsions within the Gln2 and Val4 residues to the β-sheet region of Ramachandran space. In contrast, the N-aminated aIle3 and aTyr5 residues exhibit greater conformational heterogeneity. This pattern was also observed in the case of di-NAP 13. To specifically parse the conformational impact of N-amination, we carried out unrestrained conventional molecular dynamics (MD) simulations on 12 and AcPHF6. Ramachandran plots for the 400 ns simulation again showed that N-amination severely restricts accessible backbone torsions of the preceding residue. We previously showed (Chem. Commun. 2015, 51, 16259) that NAPs readily engage in intraresidue C6 H-bonds between the N-NH₂ donor and the carbonyl O acceptor, even in protic solvents. Coupled with the constraint imposed on the preceding residue, the hydrazide bond thus may serve to further stabilize β-sheet-like conformations that recognize fibrillar tau.

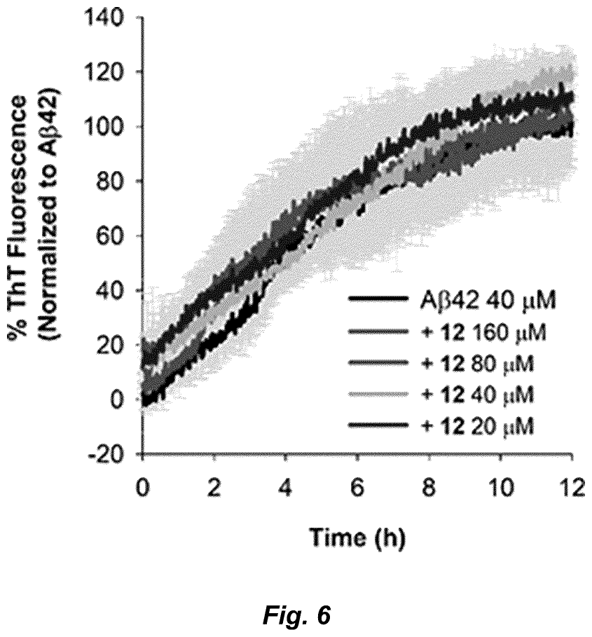

Di-NAP 12 Does Not Inhibit Aβ₄₂ Aggregation In Vitro. Many small-molecule protein aggregation inhibitors exhibit undesired promiscuity. A peptidomimetic approach to tau inhibition offers prospects for achieving selectivity over other amyloids rich in f structure. As a preliminary test, we selected our best-performing aggregation-inhibitory tau mimic, 12, and determined its effect on Aβ₄₂ aggregation in vitro (FIG. 6). Incubation of synthetic, full-length Aβ₄₂ (40 μM) in the presence of ThT and various concentrations of 12 resulted in strong fluorescence indicative of aggregation. Di-NAP 12 exhibited no inhibitory effect on Aβ₄₂ aggregation up to a 4-fold molar excess (160 μM). We similarly observed no effect on aggregation kinetics at any of the concentrations tested. Compound 12 thus exhibits in vitro selectivity for tau over Aβ₄₂, an amyloid whose parallel β-sheet assembly is also driven by a hydrophobic hexapeptide core motif.

### Macrocyclic Cross-β Epitope Mimics Targeting Tau Propagation and Seeding.

Design and synthesis of aggregation-resistant backbone-aminated derivatives of mxyl-AD. The data above support the hypothesis that mxyl-AD mimetic macrocycle can engage in native-like hydrophobic cross-b interactions as well as edge-to-edge H-bonds along the fibril axis. We next sought to employ mxyl-AD as a template for the design of tau seeding inhibitors that could potentially cap fibril growth and block propagation. This required us to incorporate a chemical modification onto mxyl-AD that would preclude its ability to self-assemble without disrupting its capacity to adopt the conformation required to bind tau. We therefore designed two backbone N-amino peptide (NAP) derivatives of our tau mimic, mxyl-NAP1 and mxyl-NAP2 (Scheme 1). Each of these analogues features a backbone amide substitution that precludes canonical b-sheet H-bonding along one edge of the macrocycle, while leaving the other edge intact to engage the tau protein. We previously demonstrated that backbone N-amination within strand regions of hairpin peptide model systems can enhance b-sheet-like conformation and convert aggregation-prone sequences into soluble, aggregation-resistant peptidomimetics. Both mxyl-NAP1 and mxyl-NAP2, which feature a single backbone N-amino group within the amyloidogenic PHF6 module, were prepared on solid support starting from Fmoc-protected N-amino dipeptides. As anticipated, both compounds exhibited enhanced solubility in DMSO and aq. PBS relative to mxyl-AD and did not form aggregates as judged by ThT assay.

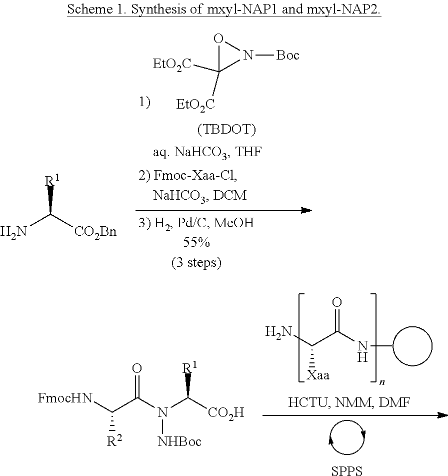

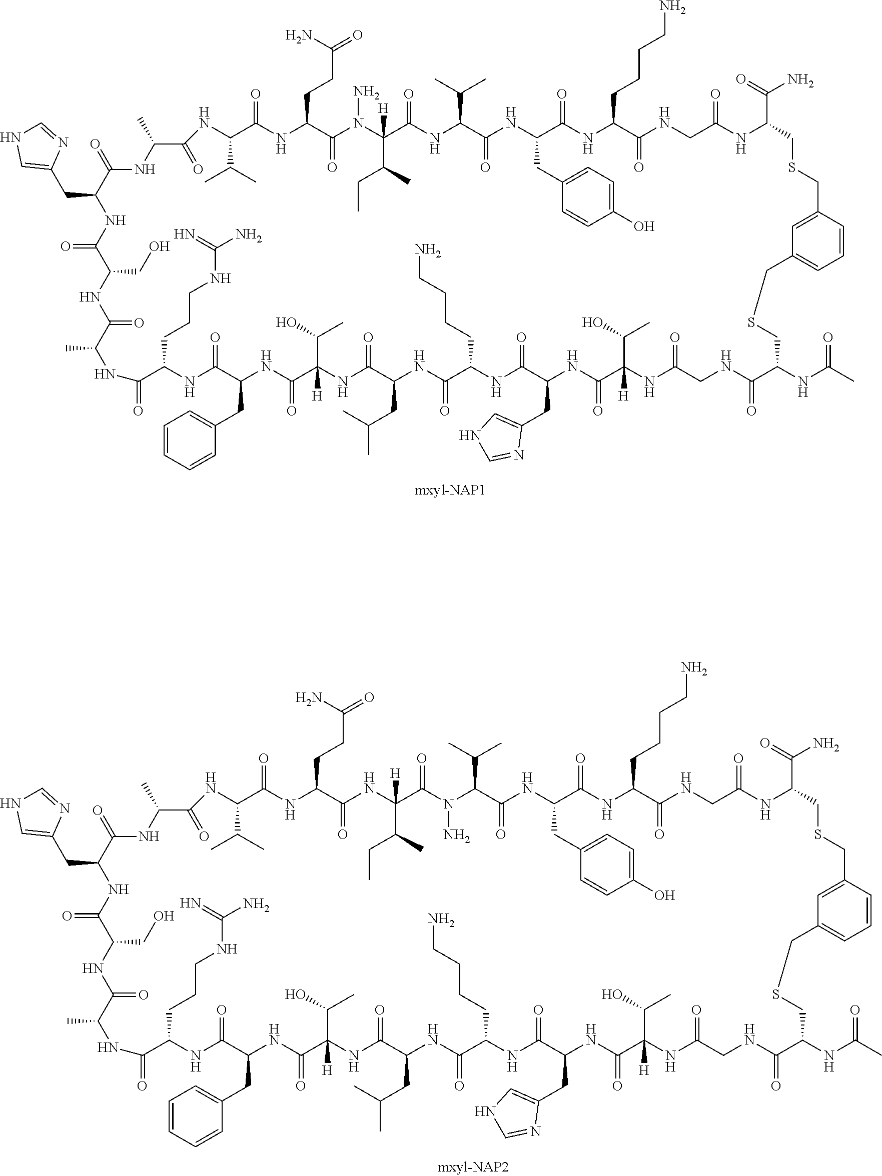

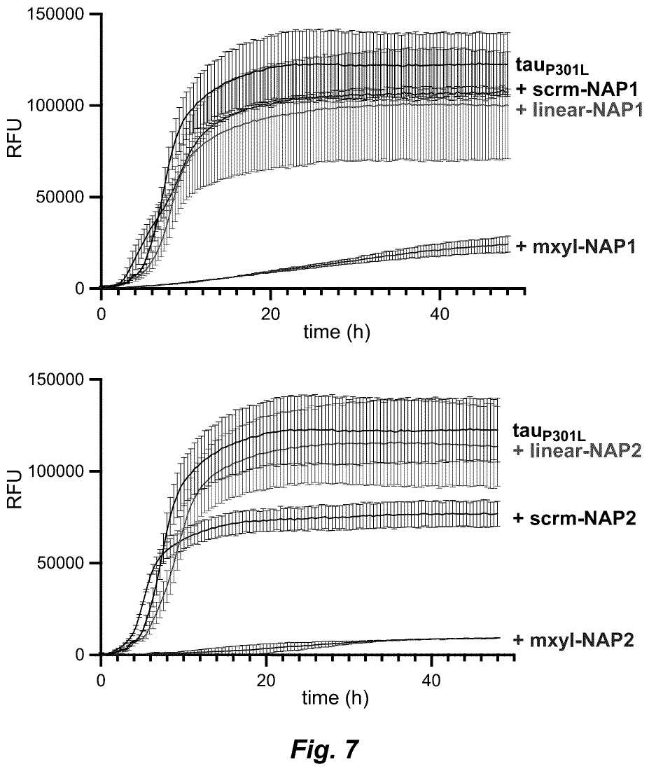

Inhibition of full-length tau aggregation by mxyl-NAPs in a ThT assay. To test whether mxyl-NAPs effectively inhibit the growth of tau fibrils, we expressed and purified the P301L mutant of 0N4R tau (tau_{P301L}) for evaluation in ThT assays. Tau_{P301L} (5 mL) aggregated upon incubation with heparin over the course of 2 days, leading to high ThT fluorescence. This fluorescence was significantly attenuated in the presence of 10 mM mxyl-NAP1 or mxyl-NAP2 (FIG. 7). We also synthesized linear and scrambled controls corresponding to each of the NAP inhibitors. In the case of mxyl-NAP1, neither of the controls (linear-NAP1 or scrm-NAP1) showed a significant effect on tau_{P301L} aggregation at 10 mM. Incubation with scrm-NAP2 led a modest reduction of tau aggregation by ThT, but was still far less effective than mxyl-NAP2.

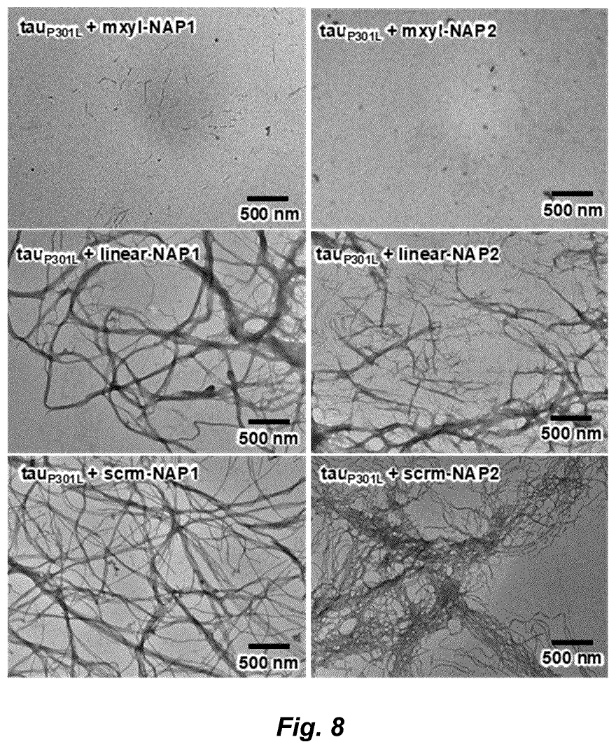

Inhibition of full-length tau fibrilization by mxyl-NAPs in TEM. To confirm the results from ThT assays, we used TEM to visualize tau_{P301L} fibrils in the absence or presence of synthesized NAPs. As expected, heparin-induced tau_{P301L} formed dense networks of mature fibrils (FIG. 8). Incubation of 5 mM tau_{P301L} in the presence of 10 mM mxyl-NAP1 showed no elongated fibrils and only sparse areas containing small protofibrliar structures. No fibrils were found in the presence of mxyl-NAP2. In contrast, linear-NAP1, scrm-NAP1, linear-NAP2, and scrm-NAP2 had little effect on the fibrilization of tau_{P301L} as judged by TEM. Each of the samples treated with these control compounds showed mature fibrils similar to those in the untreated wells. Results from TEM were thus in agreement with ThT fluorescence assays and demonstrate that the inhibitory effect of mxyl-NAPs is both macrocycle- and sequence-dependent.

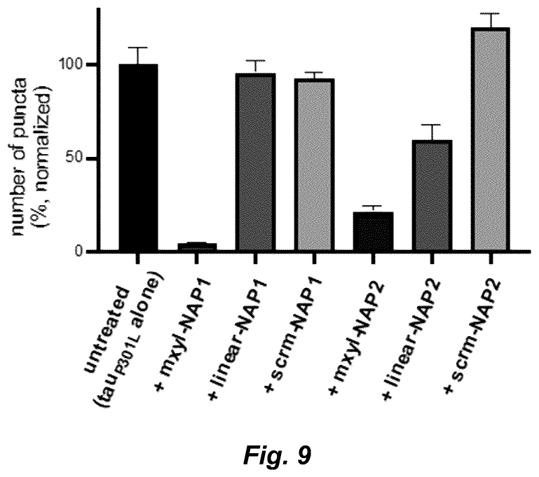

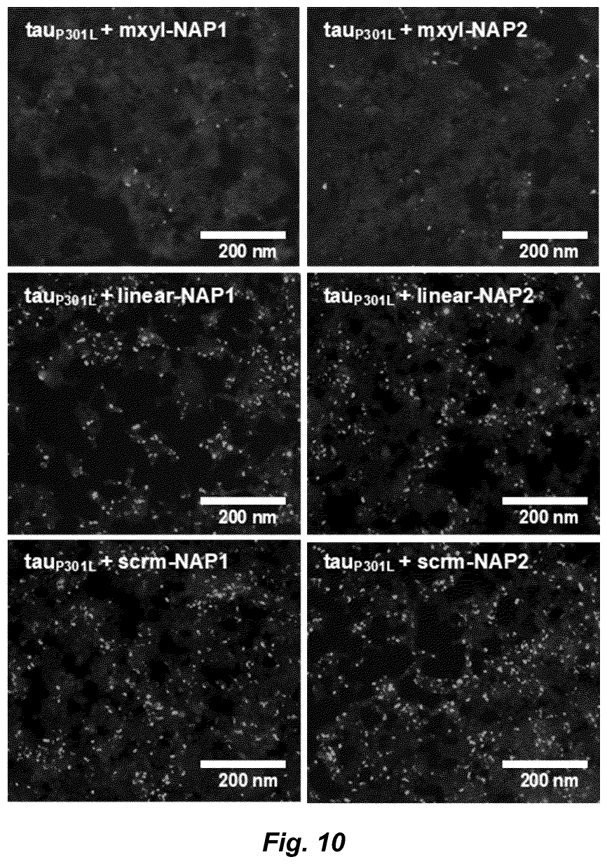

Mxyl-NAPs inhibit the seeding capacity of full-length tau in a biosensor cell assay. The ability to halt the prion-like spread of tau fibrils from diseased to healthy cells using designed molecules remains a considerable challenge. While several compounds demonstrate inhibitory activity against the aggregation of tau (or short peptides derived from tau) in vitro, smaller oligomeric forms of tau may still be able to nucleate the growth of endogenous fibrils once taken up by cells. To test whether mxyl-NAPs could block this seeding activity, we treated HEK293 biosensor cells that express a tau yellow fluorescent protein fusion (tau-RD[LM]-YFP) with tau_{P301L} incubated in the presence or absence of inhibitors. Heparin-induced fibrils of tau_{P301L} grown in the absence of mxyl-NAPs led to strong intracellular FRET signal resulting from endogenous tau-RD(LM)-YFP aggregation. Quantitative brightness threshold-based analysis of puncta within the biosensor cells provided a positive baseline for seed-competent recombinant tau fibrils. For these experiments, we found that tau_{P301L} fibrils grown for 4 days with low molecular weight heparin yielded the most active seeds (˜60,000 puncta/15,000 cells upon treatment with 190 nM tau_{P301L}). Remarkably, 95 nM of mxyl-NAP1 almost totally blocked the seeding activity of tau_{P301L} when added to the monomeric protein in the fibril growth stage (FIG. 9 and FIG. 10). Moreover, linear-NAP1 and scrm-NAP1 at the same concentration had no effect on the seeding capacity of heparin-induced tau_{P301L} as measured by fluorescent punctate counting. Mxyl-NAP2 similarly exhibited potent inhibition of tau_{P301L} seeding activity at 95 nM, with control analogues displaying modest or no activity in the biosensor cell assay.

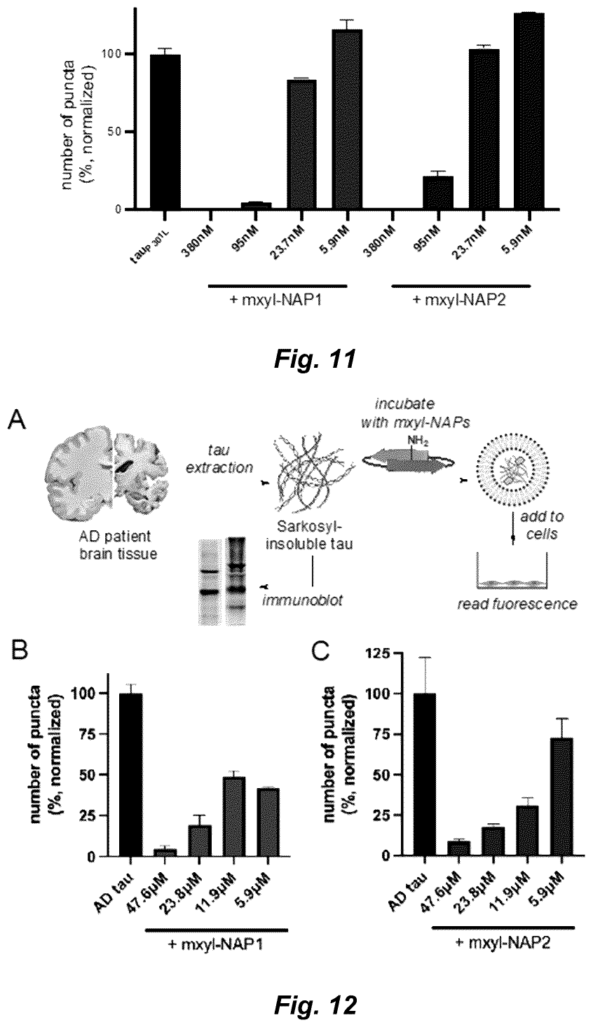

We then established the dose-dependent anti-seeding activity of both mxyl-NAP1 and mxyl-NAP2. Tau_{P301L} (190 nM) treated with either 2 equiv (380 nM) or 0.5 equiv (95 nM) of each inhibitor exhibited significantly reduced ability to seed endogenous tau-RD(LM)-YFP, while lower concentrations were less effective (FIG. 11). Data from biosensor cellular assays demonstrate that mxyl-NAPs not only impair mature tau fibril growth but also abrogate the formation of smaller seed-competent tau oligomers that may be critical for templated tau propagation across cells.

Mxyl-NAPs inhibit the seeding capacity of AD tau derived from patient samples. Given that our macrocyclic NAPs were designed based on the amyloidogenic cross-b structure within the tau AD fold, we examined whether these compounds could inhibit the seeding activity of AD tau fibrils. This assay would also assess whether mxyl-NAPs are able to cap mature tau fibrils that have already adopted the conformation observed in high-resolution structures of AD patient samples. Recapitulating the pathological folds encountered in brain tissue using recombinant protein remains a major challenge. A recent report described the characterization of an AD tau fold in fibrils grown in vitro from a truncated tau₂₉₇₋₃₉₁ construct (dGAE). We expressed and purified dGAE and confirmed its aggregation at various concentrations by ThT. However, attempts to seed endogenous tau-RD(LM)-YFP within the biosensor cells with pre-formed dGAE fibrils under various conditions were unsuccessful.

We then turned to post-mortem brain extracts of Sarkosyl-insoluble tau from AD patients (FIG. 12A). We screened 3 different patient samples for consistent seeding activity in the biosensor cells and selected the strongest patient seed extract to perform experiments with varying concentrations of mxyl-NAPs. While the untreated AD-associated tau fibrils induced ˜13,000 puncta/15,000 cells, we observed strong inhibition of seeding activity upon incubation with 23.8 and 47.6 mM of mxyl-NAP1 (FIG. 12B). Treatment of 2 mL of patient-derived AD tau extract with lower concentrations of mxyl-NAP1 (11.9 or 5.9 mM) resulted in less effective inhibition of intracellular tau-RD(LM)-YFP aggregates. Mxyl-NAP2 exhibited similar dose-dependent reduction of AD tau seeding activity, with >75% inhibitory efficacy at 23.8 mM, whereas lower concentrations were far less effective (FIG. 12C). Although we did not observe any qualitative changes in cell morphology upon treatment with mxyl-NAPs, we also sought to ensure that the compounds were not inhibiting seeding due to cytotoxicity. MTT assay using both HEK293 (biosensor) and SH-SY5Y (neuroblastoma) cells treated with mxyl-NAPs show >90% cell viability at 50 mM.

### Pharmaceutical Formulations.

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.10% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention provides therapeutic methods of treating a tauopathy in a mammal, which involve administering to a mammal having a tauopathy an effective amount of a compound or composition described herein. A mammal includes a primate, human, rodent, canine, feline, bovine, ovine, equine, swine, caprine, bovine and the like.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Methods

Solution-Phase Synthesis Notes. Unless stated otherwise, reactions were performed in flame-dried glassware under a positive pressure of argon or nitrogen gas using dry solvents. Commercial grade reagents and solvents were used without further purification except where noted. Anhydrous solvents were purchased directly from chemical suppliers. Thin-layer chromatography (TLC) was performed using silica gel 60 F254 pre-coated plates (0.25 mm). Flash chromatography was performed using silica gel (60 μm particle size). Reaction progress was judged by TLC analysis (single-spot/two-solvent systems) using a UV lamp, CAM (ceric ammonium molybdate), ninhydrin, or basic KMnO₄ stain(s) for detection purposes. NMR spectra were recorded on a 500 or 800 MHz spectrometer. Proton chemical shifts are reported as δ values relative to residual signals from deuterated solvents (CDCl₃, DMSO-d₆, and D₂O).

General Procedure for N-Amino Dipeptide Synthesis. To a solution of amino benzyl ester (HCl salt, 1.0 equiv) in a biphasic mixture of THF and sat. aq. NaHCO₃ (1:1), 2-(tert-butyl)-3,3-diethyl-1,2-oxaziridine-2,3,3-tricarboxylate (1.1 equiv) was added, and the reaction mixture was allowed to stir at rt for 4 h. The reaction was diluted with EtOAc and the aqueous layer drained. The organic layer was washed with additional water, then dried over anhydrous Na₂SO₄, filtered, and concentrated. Purification by flash chromatography over silica gel (15-50% EtOAc/hexanes) afforded the hydrazino ester as a clear oil (75-95% yield).

A solution of Fmoc amino acid (1.2 equiv) in DCM was treated with 1-chloro-N,N,2-trimethyl-1-propenylamine (1.6 equiv) and stirred for 10 min. The solution was then transferred into a flask containing a mixture of the hydrazino ester prepared above (1 equiv) and NaHCO₃ (3 equiv) in DCM. The reaction was stirred for 6 h and quenched with water. The organic layer was collected and the aq. phase extracted with additional DCM. The combined organic layers were dried over anhydrous Na₂SO₄, filtered, and concentrated. Purification by silica gel flash chromatography (15-75% EtOAc/hexanes) afforded the protected N-amino dipeptide as an off-white solid (73-82% yield).

To a solution of the protected N-amino dipeptide prepared above (1.0 equiv) in EtOAc was added 10% Pd/C (150 mg/mmol), and the mixture was stirred under a H₂ atmosphere at rt for 6 h. The reaction was diluted with additional EtOAc, filtered through Celite, and concentrated. Purification by flash chromatography (25-100% EtOAc/hexanes) afforded the aminated carboxylic acid as a white solid (68-94%).

Solid-Phase Synthesis of NAPs. The SPPS was carried out on CEM's Liberty Blue peptide synthesizer with an Fmoc-capped polystyrene Rink amide MBHA resin (100-200 mesh, 0.05-0.15 mmol scale). The following amino acid derivatives suitable for Fmoc SPPS were used: Fmoc-Gln(Trt)-OH, Fmoc-Asn(Trt)-OH, Fmoc-Tyr(tBu)-OH, Fmoc-Lys(Boc)-OH, Fmoc-Val-OH, and Fmoc-Ile-OH. The dry resin was washed with DMF three times and allowed to swell in DMF for 5 min at elevated temperature prior to use. All reactions were carried out using gentle agitation. Fmoc deprotection steps were carried out by treating the resin with a solution of 20% piperidine/DMF (5 min×2). Coupling of Fmoc-protected amino acids as well as Fmoc-(N′-Boc)-hydrazino dipeptide acids was effected using 5 equiv of HCTU (0.5 M in DMF), 10 equiv. of NMM (1.0 M in DMF), and 5 equiv of the carboxylic acid in DMF at 50° C. (10 min). After each reaction, the resin was washed with DMF two times and DCM two times. N-Terminal acetylation was carried out with 20 equiv of Ac₂O and 40 equiv of pyridine at rt in DCM (10 min, two times), followed by washing with DCM five times. Peptides were cleaved from the resin by incubating with gentle stirring in 2 mL of 95:2.5:2.5 TFA/H₂O/TIPS at rt for 2 h. The cleavage mixture was filtered, and the resin was rinsed with an additional 1 mL of cleavage solution. The filtrate was treated with 8 mL of cold Et₂O to induce precipitation. The mixture was centrifuged, and the supernatant was removed. The remaining solid was washed two more times with Et₂O and dried under vacuum. Peptides were analyzed and purified on C12 RP-HPLC columns (preparative: 4μ, 90 Å, 250×21.2 mm; analytical: 4μ, 90 Å, 150×4.6 mm) using linear gradients of MeCN/H₂O (with 0.1% formic acid), then lyophilized to afford white powders. All peptides were characterized by LC-MS (ESI), HRMS (ESI-TOF), and ¹H NMR. Analytical HPLC samples for all purified peptides were prepared as 1 mM in MeCN. Linear gradients of MeCN in H₂O (0.1% formic acid) were run over 20 or 12 min, and the spectra are provided for λ=220 nm.

tau_{P301L} Expression and Purification. Human tau_{P301L} (0N4R) was cloned into pET28b with an N-terminal His₆ tag. Briefly, transformed BL21 (DE3) cells were grown in LB+kanamycin medium at 37° C. until OD₆₀₀ reached 0.8 and was then induced with 1 mM IPTG overnight at 16° C. Cells were then harvested, resuspended, and lysed by probe sonication in the lysis buffer containing 20 mM Tris, 500 mM NaCl, 10 mM imidazole, and Roche cOmplete protease inhibitor cocktail, with the pH adjusted to 8.0. The lysate was then boiled for 20 min in a water bath, and the debris was pelleted by centrifugation at 20,000 g for about 40 min at 4° C. The supernatant obtained was then injected into a 5 mL IMAC Ni-charged affinity column and eluted over a gradient of 10-200 mM imidazole. Eluted tau-containing fractions were further purified using GE HiPrep 16/60 Sephacryl S-200 high-resolution size exclusion chromatography columns in a storage buffer containing 20 mM Tris, 150 mM NaCl, and 1 mM DTT, with the pH adjusted to 7.6. The purity of the protein was confirmed by SDS-PAGE analysis, and the concentration was estimated using BCA assay.

ThT Fluorescence Aggregation Assay. Recombinant tau_{P301L} (10 μM final concentration) and NAP inhibitors (20 μM final concentration) were mixed in an aggregation buffer (100 mM sodium acetate, 10 μM ThT, 10 μM heparin, 2 mM DTT, and 0.5% DMSO, pH: 7.4) in a 96-well clear bottom black plate with a final reaction volume of 200 μL. The plate was then sealed with a clear sealing film and allowed to incubate at 37° C. with continuous shaking in a Biotek Synergy H1 microplate reader. An automated method was used to carry out ThT fluorescence measurements at an excitation wavelength of 444 nm and an emission wavelength of 485 nm at an interval of every 5 min for 48 h. Experiments were carried out in technical replicates on at least 2 different days. Every experiment included control wells that lacked tau_{P301L}, heparin, or NAPs. The average signal from control wells lacking tau was used to subtract the background fluorescence, and the average of the first and last 10 data points of tau+heparin (no inhibitor) wells, after blank subtraction, was used to normalize the data. All data plots were generated with SigmaPlot.

Transmission Electron Microscopy. For analyzing fibrils by TEM, aggregation was carried out under conditions similar to the assay performed above but with ThT excluded and a final reaction volume of 100 μL. Samples were incubated in a microcentrifuge tube for 4 days at 37° C. with a mixing speed of 100 rpm. A 10 μL aliquot of the sample was then applied to 400-meshed formvar-/carbon-coated copper grids and negative-stained with 2% uranyl acetate. Micrographs were obtained on a JEOL 2011 transmission electron microscope at 200 kV.

Cellular Seeding Assay. The tau_{P301L} was diluted to a final concentration of 10 PM in an aggregation buffer containing 100 mM sodium acetate, 10 μM heparin, and 2 mM DTT, pH: 7.4. The protein was incubated in a microcentrifuge tube for 4 days at 37° C. with a shaking speed of 100 rpm. Control vials included those wherein (1) buffer was added in place of tau_{P301L} and (2) buffer was added in place of heparin.

HEK293 cells stably expressing tau-RD (LM)-YFP were cultured in DMEM containing 10% FBS, 1% penicillin/streptomycin, and 1% Glutamax in a 75 cm² cell culture flask under 5% CO₂ at 37° C. For each experiment, cells were plated at a density of 15,000 cells/well in 96-well tissue culture plates.

For seeding by monomeric tau, freshly purified recombinant tau_{P301L} was co-incubated with heparin and NAPs for 4 days in an aggregation buffer at 37° C. (see the above section). Following incubation, the reaction mixture was diluted in the low-serum Opti-MEM medium, mixed with Lipofectamine 2000 in a 20:1 ratio (complex/Lipofectamine) and allowed to incubate for an additional 20 min at rt. A mixture of 0.19 μM tau+1.9 μM or 0.009 μM inhibitors (final concentrations) was added to the cells. Cells were incubated for an additional 48 h before taking measurements on a BioTek Cytation 5 cell imager and a microplate reader. 10×10 pictures/well were taken at 20× magnification under an FITC channel, and the punctate counting was carried out using built-in software. Each data set was collected from technical replicates on at least 2 different days. Every experiment included control wells (no tau, no heparin, and no NAP). All data plots were generated with SigmaPlot. Error bars shown are standard deviation from technical replicates.

For seeding experiments with fibrillar tau, recombinant tau_{P301L} fibrils were prepared as described above (see the section on fibril formation) and sonicated for 3 min prior to use in this assay. In a reaction volume of 40, 8 μL of fibrils was diluted with 31 μL of low-serum Opti-MEM medium and then mixed with 1 μL of NAPs (DMSO concentration was constant across various concentrations of inhibitors). The reaction mixture was then allowed to incubate at 37° C. for 36 h, then mixed with 2 μL of Lipofectamine 2000, and further incubated for 20 min at rt. A 10 μL aliquot of this mixture was then added to 90 μL of cells (15,000 cells/well). Cells were incubated for additional 48 h before taking measurements on a BioTek Cytation 5 cell imager and a microplate reader. 10×10 pictures/well were taken at 20× magnification under an FITC channel, and the punctate counting were carried out using built-in software. Each data set were collected from technical replicates on at least 2 different days. Every experiment had a tau control well (no tau but rest all), a heparin control well (no heparin but rest all), and a Tau alone well (no inhibitors but rest all). Every experiment included control wells (no tau, no heparin, and no NAP). All data plots were generated with SigmaPlot. IC₅₀ values were calculated by fitting the data set using the sigmoidal logistic four-parameter equation. Error bars represent standard deviation from technical replicates.

Human Serum Stability Assay. The stability of NAPs in 25% human serum (MilliporeSigma) was assessed by HPLC. The reaction was started by adding NAPs at a final concentration of 500 μM in pre-warmed serum. The mixture was incubated at 37° C. for 24 h. A 100 μL aliquot of the reaction mixture was taken out at 0, 1, 4, and 24 h and was mixed with an equal volume of 20% trichloroacetic acid and incubated at 4° C. for 15 min to precipitate serum proteins. After centrifugation at 12,000 rpm for 10 min, the supernatant was collected and mixed with an internal standard (1 mg/mL Cbz-Tyr-OH dissolved in MeCN) and stored at −20° C. Samples were then analyzed by LC-MS, and the percentage of peptide remaining was calculated by integrating peaks.

MTT Cell Viability Assay. MTT cell viability assays were carried out on both HEK293 cells stably expressing tau-RD (LM)-YFP and SH-SY5Y cells. Cells were cultured in the DMEM/F12 complete medium containing 10% FBS, 1% penicillin/streptomycin, and 1% Glutamax in a 75 cm² cell culture flask under 5% CO₂ at 37° C. Cell viability was determined using the MTT reduction assay. Briefly, 15,000 cells/well were plated in a 96-well tissue culture plate and were allowed to incubate overnight in a CO₂ incubator. The medium was aspirated and the NAP inhibitor prepared in complete medium was added at a given final concentration. The plate was then allowed to incubate for additional 48 h in a CO₂ incubator, and the medium was aspirated again and replaced with 0.5 mg/mL MTT prepared in complete medium and incubated for additional 3 h. The medium was then replaced with DMSO to dissolve formazan crystals, and the absorbance was measured at 570 nm using a Synergy H1 microplate reader. Each data set was collected from technical replicates on at least 2 different days.

NOESYNMR Acquisition Parameters. Purified peptides 12 and 13 were dissolved in 9:1 H₂O/D₂O or DMSO-d₆. The final peptide concentration was 1 mM, determined by mass. Data were collected at 25° C. on a 500 MHz Bruker ASCEND, 11.74 T; narrow bore, 54 mm; BOSS-3 36 shim system; BSMS shim and digital lock control units with a 5 mm direct detect SMART probe (¹H/¹³C/¹⁵N with Z axis PFG), or an 800 MHz AVANCE II with UltraStabilized and UltraShield (18.79 T), 54 mm bore, a BOSS-2 34 shim system, and a 5 mm broad band (BBO) 15N-31P, 1H decoupling, Z-axis PFG. The TOCSY used a mixing time of 80 ms, and the ROESY had a mixing time of 200 ms. In the f2 direction, the TOCSY and ROESY had 2048 complex points collected, and in the f1 direction, 512 complex points were collected. Watergate 3-9-19 was used for solvent suppression where appropriate. Bruker TopSpin 4.0 or Mestrenova 10.0 software was used to process the data, and Gaussian functions were used before Fourier transformation.

MD Simulations. Simulated annealing was done with NOE distance and dihedral restraints. The simulated annealing protocol includes the following steps: (1) initial structures of 12 and 13 were prepared using Maestro (version 12.6.149, Schrödinger, LLC). (2) Each initial structure was first energy minimized in vacuum. (3) Next, beginning with the minimized structure, 100 replicas were generated with different initial velocities, and each replica was heated from 300 to 800 K in 100 ps and simulated at 800 K for another 100 ps. (4) After annealing, each replica was solvated. The dimensions of the box were chosen such that the distance between the walls of the box and any atom of the compound was at least 1.0 nm. Minimal counterions were added to neutralize the net charge of the system. The entire system was then energy minimized using the steepest descent algorithm to remove any bad contacts. (5) Next, the system underwent a 500 ps NVT equilibration at 300 K. (6) Lastly, the system was annealed from 300 to 500 K and then subsequently down to 5 K over 1 ns in an NPT ensemble (the temperature was increased from 300 to 500 K in the first 100 ps, maintained at 500 K for 100 ps, decreased to 300 K in the following 500 ps, maintained at 300 K for 100 ps, and then decreased to 5 K in the last 200 ps). (7) After all the simulation steps, the final frames from each of the 100 trajectories were used for the analysis.

GROMACS 4.6.7 suite with the OPLS2005 force field with a TIP4P water model was used for the simulations. The topology file for each compound was generated using the Schrödinger utility ffld_server and converted to the GROMACS format using the ffconv.py script. Throughout the simulated annealing protocol, NOE-derived distance restraints were applied to the compound with a force constant of 10,000 kJ·mol⁻¹·nm⁻². The force constant for dihedral restraints was 1000 kJ·mol⁻¹·rad⁻². The temperature was regulated using a v-rescale thermostat, with a coupling time constant of 0.1 ps. The pressure was regulated using a Berendsen barostat, with a time coupling constant of 2.0 ps and an isothermal compressibility of 4.5×10⁻⁵ bar⁻¹. The leapfrog algorithm with an integration time step of 2 fs was used to evolve the dynamics of the system. The linear constraint solver (LINCS) algorithm was used to constrain all bonds containing hydrogens to the equilibrium bond lengths. For simulations in vacuum, the cutoffs of all non-bonded (electrostatic and van der Waals) interactions were set to 999.0 nm, and the neighbor list was constructed only once and never updated. For simulations in solvent, all non-bonded interactions as well as neighbor searching were truncated at 1.0 nm. Long-range electrostatics beyond 1.0 nm were calculated using the particle mesh Ewald method with a Fourier spacing of 0.12 nm and an interpolation order of 4. To account for truncation of the Lennard-Jones interactions, a long-range analytic dispersion correction was applied to both energy and pressure.

Dihedral principal component analysis was performed on the backbone (ϕ, ψ) angles of residues V, Q, I, V, Y, and K of 12 and 13. The first three principal components were used for further cluster analysis. The population for each cluster was calculated. For 12, 99 structures were grouped into 18 clusters. For 13, 87 structures were grouped into 16 clusters.

Conventional MD Simulations. Conventional MD simulations were performed for AcPHF6 and 12. The initial structures were built using Maestro (version 12.6.149, Schrödinger, LLC). All MD simulations in this study were performed using the GROMACS 4.6.7 suite with the OPLS 2005 force field and the TIP4P water model. The topology file for each compound was generated using the Schrödinger utility ffld_server and converted to the GROMACS format using the ffconv.py script. Each initial structure was first energy minimized for 10,000 steps and then solvated in a cubic box of water molecules. The box size was chosen such that the distance between the compound and the box wall was at least 1.0 nm. Minimal explicit counterions were also added to neutralize the net charge of the system. With all heavy atoms restrained, the solvated system was further energy minimized for 5000 steps. With all the heavy atoms remaining restrained to their initial coordinates, a 50 ps NVT equilibration at 300 K was performed, followed by a 50 ps NPT equilibration at 300 K and 1 bar to adjust the solvent density. Then, the position restraints on heavy atoms were removed. The system underwent a further equilibration process in the NVT ensemble for 100 ps and in the NPT ensemble for 100 ps. The equilibrated system then underwent a 500 ns production run in the NPT ensemble at 300 K and 1 bar. In all the simulations, the temperature was regulated using the v-rescale thermostat with a coupling time constant of 0.1 ps. To avoid the “hot solvent/cold solute” artifacts, two separated thermostats were applied to the solvent (water and ions) and the compound. For the NPT simulations, the pressure was maintained using the isotropic Parrinello-Rahman barostat with a coupling time of 2.0 ps and a compressibility of 4.5×10⁻⁵ bar⁻¹. Bonds involving hydrogen were constrained using the LINCS algorithm. A 2 fs time step was used with the leapfrog integrator. The nonbonded interactions (Lennard-Jones and electrostatic) were truncated at 1.0 nm. Long-range electrostatic interactions were treated using the particle mesh Ewald summation method with a Fourier spacing of 0.12 nm and an interpolation order of 4. A long-range analytic dispersion correction was applied to both the energy and pressure to account for the truncation of Lennard-Jones interactions. The last 400 ns of each production run was used for further analysis.

### Example 2. Compound Characterization Data

Fmoc-Ile-(N′-Boc)aVal-OH Obtained as a white solid (54% yield over 3 steps); ¹H NMR (500 MHz, CDCl3, mixture of rotamers) δ 8.60 (s, 1H), 8.19 (s, 1H), 7.76 (d, J=7.5 Hz, 2H), 7.66-7.49 (m, 2H), 7.44-7.37 (m, 2H), 7.33-7.29 (m, 2H), 5.62-5.42 (m, 0.8H), 5.16 (m, 0.2H), 4.78-4.61 (m, 0.4H), 4.55-4.14 (m, 4.6H), 2.50-2.27 (m, 0.4H), 2.09-1.86 (m, 1.6H), 1.71-1.60 (m, 1H), 1.58-1.47 (m, 9H), 1.23-0.84 (m, 13H); ¹³C NMR (126 MHz, CDCl3, mixture of rotamers) δ 175.8, 175.7, 172.6, 171.5, 158.3, 157.5, 156.1, 155.7, 144.0, 143.6, 143.5, 141.3, 127.9, 127.8, 127.7, 127.1, 125.3, 125.2, 125.1, 125.0, 120.1, 120.0, 120.0, 84.7, 84.6, 77.4, 67.8, 67.6, 67.0, 64.1, 55.9, 54.8, 47.3, 46.9, 38.5, 38.0, 35.2, 28.3, 28.2, 27.9, 26.8, 24.6, 23.9, 23.6, 21.1, 20.1, 19.2, 19.1, 16.1, 15.8, 11.7, 10.5; HRMS (ESI-TOF) m/z [M+H]⁺ calcd for C31H42N3O7 569.3096, found 569.3121.

Fmoc-Ile-(N′-Boc)aIle-OH Obtained as a white solid (47% yield over 3 steps); ¹H NMR (500 MHz, CDCl3, mixture of rotamers) δ 8.52 (s, 0.67H), 8.07 (s, 0.13H), 7.76 (d, J=7.5 Hz, 2H), 7.65-7.50 (m, 2H), 7.45-7.37 (m, 2H), 7.35-7.28 (m, 2H), 5.57-5.43 (m, 0.9H), 5.24 (s, 0.1H), 4.83-4.64 (m, 0.25H), 4.60 (d, J=8.0 Hz, 0.75H), 4.49-4.27 (m, 3H), 4.26-4.10 (m, 2H), 2.07 (bs, 0.25H), 1.99-1.86 (m, 0.75H), 1.79-1.61 (m, 2H), 1.58-1.37 (m, 10H), 1.28-1.15 (m, 2H), 1.09-0.84 (m, 13H); ¹³C NMR (126 MHz, CDCl3, mixture of rotamers) δ 175.6, 175.2, 173.9, 172.8, 171.3, 158.2, 157.4, 156.0, 155.5, 154.2, 143.8, 143.4, 143.3, 141.2, 127.8, 127.6, 127.6, 127.5, 127.1, 127.0, 125.2, 125.1, 125.0, 124.9, 124.9, 120.0, 119.9, 119.9, 119.8, 84.5, 82.5, 67.7, 66.9, 66.1, 62.9, 62.6, 55.8, 55.3, 54.6, 47.1, 46.8, 38.5, 37.9, 35.2, 35.1, 35.0, 33.0, 28.0, 27.7, 27.2, 26.8, 26.4, 24.5, 23.7, 23.5, 15.9, 15.6, 15.4, 12.1, 11.7, 11.5, 11.2, 10.3; HRMS (ESI-TOF) m/z [M+H]⁺ calcd for C32H44N3O7 583.3252, found 583.3255.

Fmoc-Gln(Trt)-(N′-Boc)aIle-OH Obtained as a white solid (42% yield over 3 steps); ¹H NMR (500 MHz, CDCl3, mixture of rotamers) δ 8.27 (bs, 0.2H), 8.00 (bs, 0.4H), 7.80-7.70 (m, 2H), 7.65-7.52 (m, 2H), 7.39 (q, J=7.5 Hz, 2H), 7.34-7.10 (m, 17H), 6.91 (bs, 0.5H), 6.73 (bs, 0.3H), 5.99-5.87 (m, 0.25H), 5.77 (d, J=8.0 Hz, 0.6H), 4.93-4.86 (m, 0.2H), 4.80-4.51 (m, 2H), 4.45-4.30 (m, 2H), 4.27-4.17 (m, 1H), 2.49-2.03 (m, 3H), 1.99-1.58 (m, 3H), 1.51-1.09 (m, 10H), 1.01-0.80 (m, 6H); ¹³C NMR (126 MHz, CDCl3, mixture of rotamers) δ 175.2, 174.5, 172.5, 172.0, 171.8, 156.1, 156.0, 154.9, 154.4, 144.2, 144.1, 143.7, 141.3, 141.2, 128.6, 127.9, 127.6, 127.0, 125.2, 125.1, 119.9, 83.3, 82.1, 77.2, 70.9, 66.8, 63.0, 51.1, 47.2, 34.5, 34.4, 33.2, 28.9, 28.6, 28.1, 27.8, 26.6, 15.8, 15.6, 11.8, 11.6; HRMS (ESI-TOF) m/z [M+H]⁺ calcd for C50H55N4O8 840.4093, found 840.4117.

Fmoc-Val-(N′-Boc)aTyr(tBu)-OH Obtained as a white solid (45% yield over 3 steps); ¹H NMR (500 MHz, CDCl3, mixture of rotamers) δ 7.76-7.65 (m, 3H), 7.53-7.46 (m, 2H), 7.38-7.22 (m, 5H), 7.06-7.00 (m, 0.5H), 6.91 (m, 1.5H), 6.72 (d, J=8.0 Hz, 1H), 6.04 (bs, 0.3H), 5.35-5.11 (m, 1.5H), 4.41-4.05 (m, 4H), 3.99-3.89 (m, 1H), 3.34-3.19 (m, 1.5H), 2.81 (t, J=13.8 Hz, 0.5H), 2.04-1.91 (m, 0.5H), 1.70 (s, 0.5H), 1.51-1.34 (m, 9H), 1.31-1.19 (m, 4H), 1.11 (s, 6H), 0.93-0.79 (m, 6H); ¹³C NMR (126 MHz, CDCl3, mixture of rotamers) δ 175.5, 174.9, 172.7, 171.6, 157.9, 156.8, 155.9, 155.7, 154.1, 153.8, 143.8, 143.6, 143.4, 141.1, 131.8, 130.6, 130.5, 129.3, 128.5, 127.6, 126.9, 125.1, 124.3, 124.1, 119.8, 84.4, 84.2, 78.3, 67.3, 66.9, 62.4, 58.7, 55.9, 55.7, 47.0, 46.9, 46.8, 33.5, 32.6, 31.0, 29.5, 29.4, 28.6, 28.0, 27.4, 19.5, 18.0, 16.5; HRMS (ESI-TOF) m/z [M+H]⁺ calcd for C38H48N3O8 675.3514, found 675.3531.

Fmoc-Tyr(tBu)-(N′-Boc)aLys(Boc)-OH Obtained as a white solid (53% yield over 3 steps); ¹H NMR (500 MHz, CDCl3, mixture of rotamers) δ 8.63 (bs, 0.3H), 7.77 (d, J=7.5 Hz, 2H), 7.63-7.50 (m, 2H), 7.41 (t, J=7.5 Hz, 2H), 7.32 (t, J=7.4 Hz, 2H), 7.14-6.76 (m, 5H), 5.69-5.42 (m, 0.9H), 5.25 (m, 0.1H), 4.98-4.54 (m, 3H), 4.41-4.14 (m, 3H), 3.23-2.80 (m, 2H), 1.98 (bs, 0.6H), 1.81-1.65 (m, 1.3H), 1.56-1.24 (m, 35H); 13C NMR (126 MHz, CDCl3, mixture of rotamers) δ 174.5, 173.4, 173.0, 171.9, 156.2, 155.2, 154.0, 143.8, 143.5, 141.1, 130.9, 129.9, 127.5, 126.9, 125.1, 124.1, 124.0, 119.8, 84.3, 84.0, 82.8, 80.7, 79.1, 78.4, 78.3, 77.2, 67.4, 66.8, 62.3, 59.6, 59.0, 58.4, 52.6, 52.0, 47.0, 46.8, 41.2, 40.2, 39.7, 38.5, 37.6, 36.6, 33.9, 31.8, 29.6, 29.3, 28.7, 28.3, 28.0, 27.8, 23.7, 23.4; HRMS (ESI-TOF) m/z [M+H]⁺ calcd for C44H59N4O10 804.4304, found 804.4329.

Ac-aVal-Gln-Ile-Ile-Asn-Lys-NH₂ (1) (SEQ ID NO: 3). The crude peptide was purified by preparative scale RP-HPLC using a 5-95% MeCN/H2O gradient (with 0.1% formic acid). The pure peptide was obtained in 81% yield. HRMS (ESI-TOF) m/z [M+H]⁺ calcd for C34H64N11O9 770.4883, found 770.4877.

Ac-Val-Gln-aIle-Ile-Asn-Lys-NH₂ (2) (SEQ ID NO: 4). The crude peptide was purified by preparative scale RP-HPLC using a 5-95% MeCN/H2O gradient (with 0.1% formic acid). The pure peptide was obtained in 45% yield. HRMS (ESI-TOF) m/z [M+H]⁺ calcd for C34H64N11O9 770.4883, found 770.4878.

Ac-Val-Gln-Ile-aIle-Asn-Lys-NH₂ (3) (SEQ ID NO: 5). The crude peptide was purified by preparative scale RP-HPLC using a 5-95% MeCN/H2O gradient (with 0.1% formic acid). The pure peptide was obtained in 75% yield. HRMS (ESI-TOF) m/z [M+H]⁺ calcd for C34H64N11O9 770.4883, found 770.4879.

Ac-aVal-Gln-aIle-Ile-Asn-Lys-NH₂ (4) (SEQ ID NO: 6). The crude peptide was purified by preparative scale RP-HPLC using a 5-95% MeCN/H2O gradient (with 0.1% formic acid). The pure peptide was obtained in 81% yield. HRMS (ESI-TOF) m/z [M+H]⁺ calcd for C34H65N12O9 785.4992, found 785.4992.

Ac-aVal-Gln-Ile-Val-Tyr-Lys-NH₂ (5) (SEQ ID NO: 7). The crude peptide was purified by preparative scale RP-HPLC using a 5-95% MeCN/H2O gradient (with 0.1% formic acid). The pure peptide was obtained in 88% yield. HRMS (ESI-TOF) m/z [M+H]⁺ calcd for C38H65N10O9 805.4931, found 805.4930.

Ac-Val-Gln-aIle-Val-Tyr-Lys-NH₂ (6) (SEQ ID NO: 8). The crude peptide was purified by preparative scale RP-HPLC using a 5-95% MeCN/H2O gradient (with 0.1% formic acid). The pure peptide was obtained in 55% yield. HRMS (ESI-TOF) m/z [M+H]⁺ calcd for C38H65N10O9 805.4931, found 805.4925.

Ac-Val-Gln-Ile-aVal-Tyr-Lys-NH₂ (7) (SEQ ID NO: 9). The crude peptide was purified by preparative scale RP-HPLC using a 5-95% MeCN/H2O gradient (with 0.1% formic acid). The pure peptide was obtained in 88% yield. HRMS (ESI-TOF) m/z [M+H]⁺ calcd for C38H65N10O9 805.4931, found 805.4927.

Ac-Val-Gln-Ile-Val-aTyr-Lys-NH₂ (8) (SEQ ID NO: 10). The crude peptide was purified by preparative scale RP-HPLC using a 5-95% MeCN/H2O gradient (with 0.1% formic acid). The pure peptide was obtained in 38% yield. HRMS (ESI-TOF) m/z [M+H]⁺ calcd for C38H65N10O9 805.4931, found 805.4927.

Ac-Val-Gln-Ile-Val-Tyr-aLys-NH₂ (9) (SEQ ID NO: 11). The crude peptide was purified by preparative scale RP-HPLC using a 5-95% MeCN/H2O gradient (with 0.1% formic acid). The pure peptide was obtained in 99% yield. HRMS (ESI-TOF) m/z [M+H]⁺ calcd for C38H65N10O9 805.4931, found 805.4933.

Ac-aVal-Gln-aIle-Val-Tyr-Lys-NH₂ (10) (SEQ ID NO: 12). The crude peptide was purified by preparative scale RP-HPLC using a 5-95% MeCN/H2O gradient (with 0.1% formic acid). The pure peptide was obtained in 52% yield. HRMS (ESI-TOF) m/z [M+H]⁺ calcd for C38H66N11O9 820.5040, found 820.5045.

Ac-aVal-Gln-Ile-Val-aTyr-Lys-NH₂ (11) (SEQ ID NO: 13). The crude peptide was purified by preparative scale RP-HPLC using a 5-95% MeCN/H2O gradient (with 0.1% formic acid). The pure peptide was obtained in 58% yield. HRMS (ESI-TOF) m/z [M+H]⁺ calcd for C38H66N11O9 820.5040, found 820.5041.

Ac-Val-Gln-aIle-Val-aTyr-Lys-NH₂ (12) (SEQ ID NO: 14). The crude peptide was purified by preparative scale RP-HPLC using a 5-95% MeCN/H2O gradient (with 0.1% formic acid). The pure peptide was obtained in 44% yield. HRMS (ESI-TOF) m/z [M+H]⁺ calcd for C38H66N11O9 820.5040, found 820.5048.

Ac-Val-Gln-Ile-aVal-Tyr-aLys-NH₂ (13) (SEQ ID NO: 15). The crude peptide was purified by preparative scale RP-HPLC using a 5-95% MeCN/H2O gradient (with 0.1% formic acid). The pure peptide was obtained in 42% yield. HRMS (ESI-TOF) m/z [M+H]⁺ calcd for C38H66N11O9 820.5040, found 820.5039.

Ac-aVal-Gln-aIle-Val-aTyr-Lys-NH₂ (14) (SEQ ID NO: 16). The crude peptide was purified by preparative scale RP-HPLC using a 5-95% MeCN/H2O gradient (with 0.1% formic acid). The pure peptide was obtained in 28% o yield. HRMS (ESI-TOF) m/z [M+H]⁺ calcd for C38H67N12O9 835.5149, found 835.5115.

Ac-Val-Gln-ale-Val-Tyr-Lys-NH₂ (Ac-PHF6) (SEQ ID NO: 1). The crude peptide was purified by preparative scale RP-HPLC using a 5-95% o MeCN/H2O gradient (with 0.1% o formic acid). The pure peptide was obtained in 92% o yield. HRMS (ESI-TOF) m/z [M+H]⁺ calcd for C38H63N9O9 790.4822, found 790.4833.

Ac-Val-Gln-Ile-Ile-Asn-Lys-NH₂ (Ac-PHF6*) (SEQ ID NO: 2). The crude peptide was purified by preparative scale RP-HPLC using a 5-95% o MeCN/H2O gradient (with 0.1% o formic acid). The pure peptide was obtained in 50% o yield. HRMS (ESI-TOF) m/z [M+H]⁺ calcd for C34H63N10O9 755.4774, found 755.4775.

TABLE 1
¹H NMR Data for peptides 12 and 13 (8 values in ppm).
Ac-Val-Gln-aIle-Val-aTyr-Lys-NH₂ (12) (SEQ ID NO: 14) in DMSO-d₆

| | NH | α | ß | Others |
|---|---|---|---|---|
| Val | 7.85 | 4.20 | 1.94 | γ CH₃ 0.79, 0.81; Ac CH₃ 1.85 |
| Gln | 7.88 | 5.12 | 1.81, 1.60 | γ CH₂ 2.00, 2.07; δ NH₂ 7.47, 6.48 |
| aIle | — | 4.67 | 1.98 | NH₂ 4.61; γ CH₂ 0.91; γ CH₃ 1.24; δ CH₃ 0.68 |
| Val | 7.68 | 5.12 | 1.95 | γ CH₂ 0.73 |
| aTyr | — | 5.24 | 3.01 | NH₂ 4.51; δ CH₂ 7.03; ϵ CH₂ 6.60 |
| Lys | 8.19 | 4.14 | 1.63 | γ CH₂ 1.25; δ CH₂ 1.47; ϵ CH₂ 2.68; C-term NH₂ 7.28, 7.02 |

Ac-Val-Gln-aIle-Val-aTyr-Lys-NH₂ (12) (SEQ ID NO: 14) in 9:1 H₂O:D₂O

| | NH | α | ß | Others |
|---|---|---|---|---|
| Val | 8.13 | 4.09 | 2.04 | γ CH₃ 0.93; Ac CH₃ 2.05 |
| Gln | 8.40 | 5.37 | 2.05, 1.86 | γ CH₂ 2.31, 2.35; δ NH₂ 7.54, 6.89 |
| aIle | — | 4.59 | 2.07 | NH₂ 4.66; γ CH₂ 0.85, 1.02; γ CH₃ 1.35; δ CH₃ 0.69 |
| Val | 8.23 | 5.16 | 5.16 | γ CH₂ 0.84 |
| aTyr | — | 5.32 | 5.32 | NH₂ 4.65; δ CH₂ 7.19; & CH₂ 6.83 |
| Lys | 8.44 | 4.24 | 4.24 | γ CH2 1.39; δ CH₂ 1.68; ϵ CH₂ 2.98; C-term NH₂ 7.32, 7.12 |

Ac-Val-Gln-Ile-aVal-Tyr-aLys-NH₂ (13) (SEQ ID NO: 15) in DMSO-d₆

| | NH | α | ß | Others |
|---|---|---|---|---|
| Val | 8.16 | 4.07 | 2.05 | γ CH₃ 0.95; Ac CH₃ 2.06 |
| Gln | 8.47 | 4.38 | 2.02, 1.92 | γ CH₂ 2.34; δ NH₂ 7.23, 6.74; δ NH₂ 7.54, 6.88 |
| Ile | 8.21 | 5.26 | 1.71 | γ CH₂ 1.14, 1.44; γ CH₃ 0.83; δ CH₃ 0.53 |
| aVal | N/A | 4.62 | 2.24 | NH₂ 4.49; γ CH₂ 0.82, 0.94 |
| Tyr | 8.63 | 5.53 | 3.12, 2.79 | δ CH₂ 7.20; ϵ CH₂ 6.82 |
| aLys | N/A | 4.98 | 1.93 | NH₂ 4.56; γ CH₂ 1.36, 1.30; δ CH₂ 1.71; ϵ CH₂ 2.99; |
| | | | | C-term NH₂ 7.14, 6.85 |

Ac-Val-Gln-Ile-aVal-Tyr-aLys-NH2 (13) (SEQ ID NO: 15) in 9:1 H₂O:D₂O

| | NH | α | ß | Others |
|---|---|---|---|---|
| Val | 7.92 | 4.16 | 1.95 | γ CH₃ 0.82, 0.84; Ac CH₃ 1.86 |
| Gln | 8.09 | 4.24 | 1.82, 1.67 | γ CH₂ 2.06; δ NH₂ 7.23, 6.74 |
| Ile | 7.56 | 5.28 | 1.69 | γ CH₂ 1.37, 1.00; γ CH₃ 0.63; δ CH₃ 0.75 |
| aVal | N/A | 4.65 | 2.17 | NH₂ 4.57; γ CH₂ 0.70, 0.89 |
| Tyr | 8.20 | 5.27 | 2.91, 2.67 | δ CH₂ 6.59; ϵ CH₂ 7.01 |
| aLys | N/A | 4.89 | 1.71, 1.84 | NH₂ 4.65; γ CH₂ 1.27, 1.13; δ CH₂ 1.51; ϵ CH₂ 2.70; |
| | | | | C-term NH₂ 7.47, 7.17 |

### Example 3. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| | mg/tablet |
|---|---|
| (i) Tablet 1 | |
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| | mg/mL |
|---|---|
| (iv) Injection 1 (1 mg/mL) | |
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | |
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| | wt. % |
|---|---|
| (vii) Topical Gel 1 | |
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |
| (viii) Topical Gel 2 | |
| ‘Compound X’ | 5% |
| Methylcellulose | 2% |
| Methyl paraben | 0.2% |
| Propyl paraben | 0.02% |
| Purified water | q.s. to 100 g |
| (ix) Topical Ointment | |
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | |
| ‘Compound X’ | 5% |
| White bees wax | 10% |
| Liquid paraffin | 30% |
| Benzyl alcohol | 5% |
| Purified water | q.s. to 100 g |
| (xi) Topical Cream 2 | |
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. An N-amino peptide comprising a paired helical filament hexapeptide (PHF6) wherein one or more amino acid moieties of the PHF6 have an amide nitrogen atom along the hexapeptide backbone that is N-aminated, wherein the N-aminated amide nitrogen atom is hydrazide moiety NNH₂.

2. The N-amino peptide of claim 1 comprising Formula I: 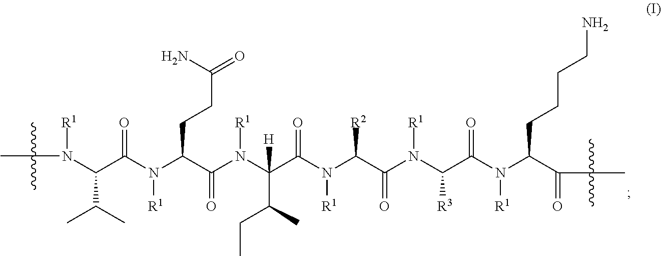
wherein
each R¹ is independently H or NH₂ wherein at least one R¹ is NH₂;
R² is isoleucine or valine; and
R³ is asparagine or tyrosine.

3. The N-amino peptide of claim 1 wherein the PHF6 comprises -Val-Gln-Ile-Val-Tyr-Lys-(VQIVYK) (SEQ ID NO: 1) or -Val-Gln-Ile-Ile-Asn-Lys-(VQIINK) (SEQ ID NO: 2).

4. The N-amino peptide of claim 1 wherein one, two, or three amino acid moieties of the PHF6 have an amide nitrogen atom along the hexapeptide backbone that is N-aminated, wherein the N-aminated amide nitrogen atom is the hydrazide moiety NNH₂.

5. The N-amino peptide of claim 1 wherein the N-amino peptide has terminal ends that terminate with an amide moiety.

6. The N-amino peptide of claim 1 wherein the N-amino peptide is:
(1)
(SEQ ID NO: 3)
Ac-aVal-Gln-Ile-Ile-Asn-Lys-NH₂;
(2)
(SEQ ID NO: 4)
Ac-Val-Gln-aIle-Ile-Asn-Lys-NH₂;
(3)
(SEQ ID NO: 5)
Ac-Val-Gln-Ile-aIle-Asn-Lys-NH₂;
(4)
(SEQ ID NO: 6)
Ac-a Val-Gln-aIle-Ile-Asn-Lys-NH₂;
(5)
(SEQ ID NO: 7)
Ac-aVal-Gln-Ile-Val-Tyr-Lys-NH₂;
(6)
(SEQ ID NO: 8)
Ac-Val-Gln-aIle-Val-Tyr-Lys-NH₂;
(7)
(SEQ ID NO: 9)
Ac-Val-Gln-Ile-aVal-Tyr-Lys-NH₂;
(8)
(SEQ ID NO: 10)
Ac-Val-Gln-Ile-Val-aTyr-Lys-NH₂;
(9)
(SEQ ID NO: 11)
Ac-Val-Gln-Ile-Val-Tyr-aLys-NH₂;
(10)
(SEQ ID NO: 12)
Ac-aVal-Gln-aIle-Val-Tyr-Lys-NH₂;
(11)
(SEQ ID NO: 13)
Ac-aVal-Gln-Ile-Val-aTyr-Lys-NH₂;
(12)
(SEQ ID NO: 14)
Ac-Val-Gln-aIle-Val-aTyr-Lys-NH₂;
(13)
(SEQ ID NO: 15)
Ac-Val-Gln-Ile-aVal-Tyr-aLys-NH₂;
or
(14)
(SEQ ID NO: 16)
Ac-aVal-Gln-aIle-Val-aTyr-Lys-NH₂;
wherein
the prefix “a” is an NH₂ moiety on an N-aminated amide nitrogen of the respective amino acid moiety of the N-amino peptide (1-14);
Ac is the acetylated N-terminus of the N-amino peptide (1-14); and
NH₂ is the amidated C-terminus of the N-amino peptide (1-14).

7. The N-amino peptide of claim 1 wherein the N-amino peptide comprises the peptide sequence:
(SEQ ID NO: 17)
-Tyr-His-Lys-Leu-Thr-Phe-Arg-^{D}Ala-Ser-His-^{D}Ala-
Val-Gln-Ile-Val-Tyr-Lys-(THKLTFR^{D}ASH^{D}AVQIVYK);
or
(SEQ ID NO: 18)
-Tyr-His-Lys-Leu-Thr-Phe-Arg-^{D}Ala-Ser-His-^{D}Ala-
Val-Gln-Ile-Ile-Asn-Lys-(THKLTFR^{D}ASH^{D}AVQIINK)

8. The N-amino peptide of claim 7 wherein the N-terminus of the peptide sequence comprises the dipeptide -Cys-Gly-, and the C-terminus of the peptide sequence comprises the dipeptide -Gly-Cys-.

9. A macrocyclic peptide comprising:
the N-amino peptide of claim 1;
a second hexapeptide configured for forming a cross-beta structure;
a beta-arc configured for an antiparallel beta-hairpin turn wherein the beta-arc is covalently linked at one end to the PHF6 and the beta-arc is covalently linked at another end to the second hexapeptide; and
a di-cysteine linker (DCL) wherein the di-cysteine linker is covalently bonded to the sulfur atoms of two cysteine moieties, wherein one cysteine moiety is conjugated via a glycine moiety to the PHF6 and the second cysteine moiety is conjugated via another glycine moiety to the second hexapeptide to complete the macrocycle of the macrocyclic peptide.

10. The macrocyclic peptide of claim 9 wherein the second hexapeptide is:
(SEQ ID NO: 19)
-Thr-His-Lys-Leu-Thr-Phe-(THKLTF);
(SEQ ID NO: 20)
-Gln-Val-Glu-Val-Lys-Ser-(QVEVKS);
or
(SEQ ID NO: 21)
-Leu-Asp-Leu-Ser-Asn-Val-(LDLSNV).

11. The macrocyclic peptide of claim 9 wherein the beta-arc is the peptide moiety:
(SEQ ID NO: 22)
-Arg-^{D}Ala-Ser-His-^{D}Ala-(R^{D}ASH^{D}A).

12. The macrocyclic peptide of claim 9 wherein the macrocyclic peptide has a C-terminus and an N-terminus, wherein both the C-terminus and the N-terminus terminate with an amide moiety.

13. The macrocyclic peptide of claim 9 wherein the di-cysteine linker (DCL) is

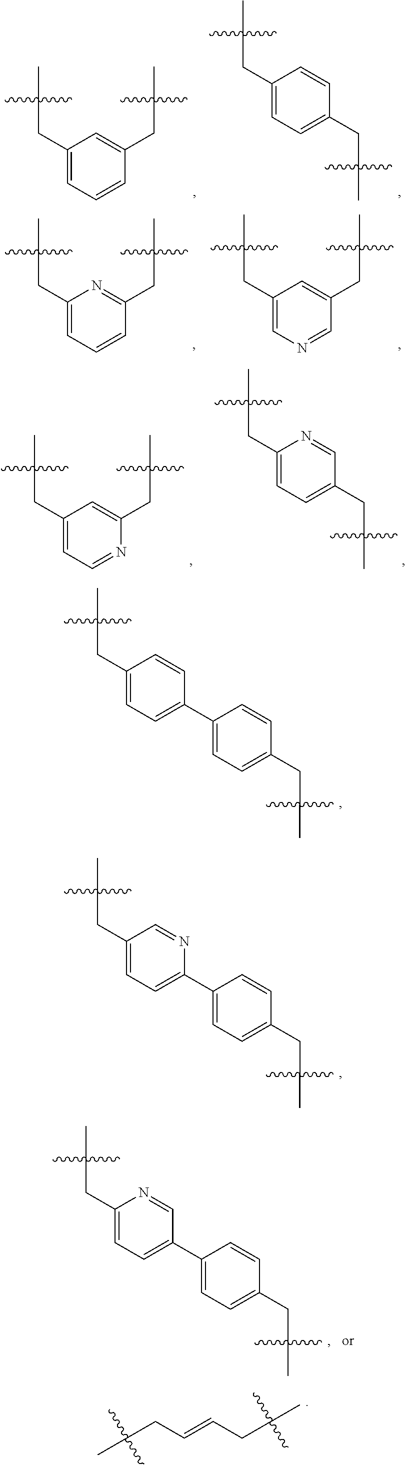

14. The macrocyclic peptide of claim 9 represented by Formula II: 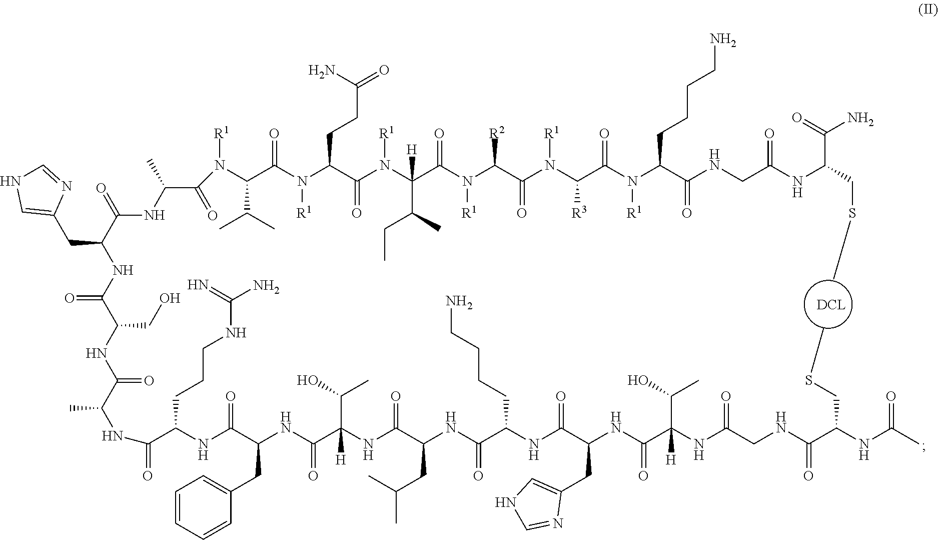
wherein
each R¹ is independently H or NH₂ wherein at least one R¹ is NH₂;
R¹ is isoleucine or valine;
R³ is asparagine or tyrosine; and
DCL is the di-cysteine linker.

15. The macrocyclic peptide of claim 14 wherein DCL is

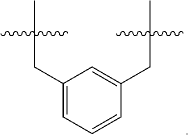 [SMILES: *Cc1cccc(C*)c1]

16. The macrocyclic peptide of claim 9 wherein the macrocyclic peptide is:

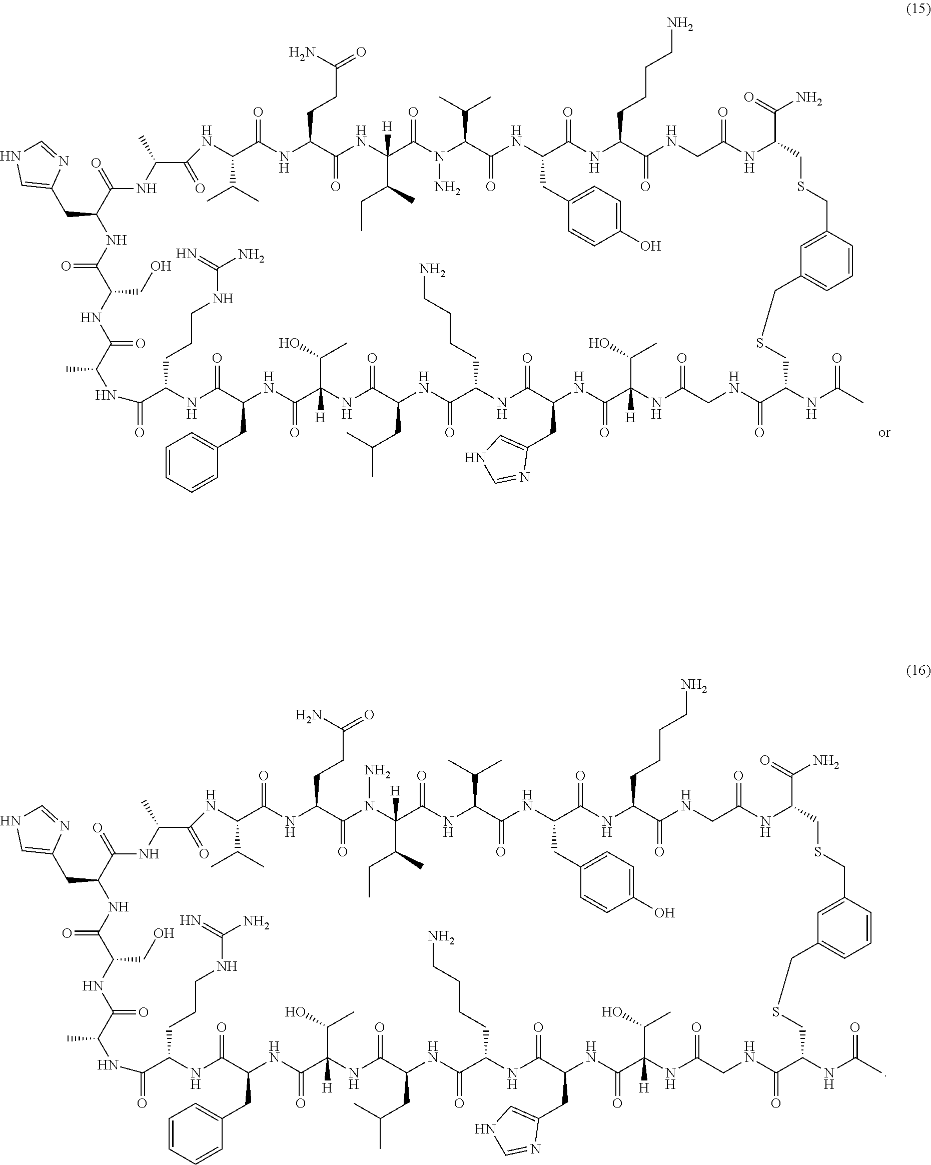

17. A method for inhibiting tau fibrilization comprising contacting an effective amount of an N-amino peptide of claim 1 and tau proteins comprising pathogenic tau fibrils, wherein the N-amino peptide blocks cellular transmission of pathogenic tau fibrils to the tau proteins and inhibits tau fibrilization of the tau proteins caused by the pathogenic tau fibrils.

18. The method of claim 17 wherein an effective amount of the N-amino peptide is about 1 micromolar to about 100 micromolar.

19. The method of claim 17 wherein the N-amino peptide is a macrocyclic peptide represented by Formula II: 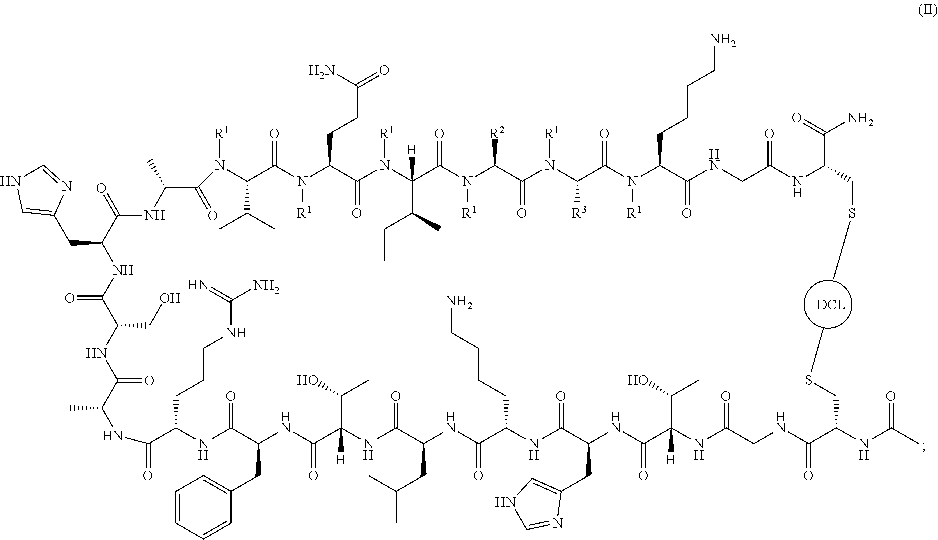
wherein
each R¹ is independently H or NH₂ wherein at least one R¹ is NH₂;
R² is isoleucine or valine;
R³ is asparagine or tyrosine; and
DCL is:

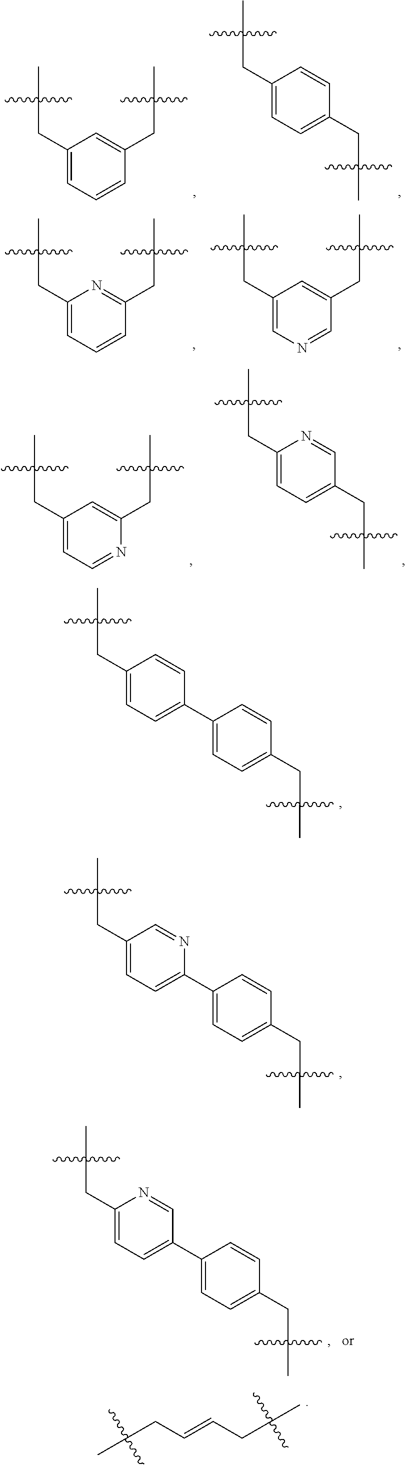

20. The method of claim 19 wherein the macrocyclic peptide is: [chemical structure]
